(19) United States
(12) Patent Application Publication
(10) Pub. No.: US 2024/0148776 A1
(43) Pub. Date: May 9, 2024

(54) TUMESCENT SOLUTION WITH B VITAMINS

(71) Applicant: HK Pharma, San Clemente, CA (US)

(72) Inventors: Jeffrey Alan Klein, San Juan Capistrano, CA (US); Paytra Alan Klein, Newport Beach, CA (US); Bram Alan Klein, Newport Beach, CA (US)

(21) Appl. No.: 18/501,539

(22) Filed: Nov. 3, 2023

Related U.S. Application Data
(60) Provisional application No. 63/382,350, filed on Nov. 4, 2022.

(51) Int. Cl. A61K 31/714; A61K 9/00; A61K 9/08; A61K 31/137; A61K 31/167; A61K 31/4025; A61K 31/522; A61K 31/546; A61K 31/573; A61P 23/02; A61P 25/02; A61P 31/22

(52) U.S. Cl. CPC A61K 31/714; A61K 9/0019; A61K 9/08; A61K 31/137; A61K 31/167; A61K 31/4025; A61K 31/522; A61K 31/546; A61K 31/573; A61P 23/02; A61P 25/02; A61P 31/22

(57) ABSTRACT

A tumescent solution including: a vasoconstrictor; a B vitamin and a pharmaceutically acceptable carrier. In some examples, the tumescent solution further includes a drug or therapeutic agent dissolved in the tumescent solution. Some examples relate to a method of subcutaneous delivery of a drug or therapeutic agent to a subject including subcutaneously administering to said subject a tumescent solution as disclosed herein. In some examples, infiltration of the tumescent solution achieves both prolonged local drug or therapeutic agent concentration within a tumescent subcutaneous tissue as well as a prolonged slow constant systemic absorption of drugs from the tumescent tissue into a systemic circulation. In some examples, the tumescent solution reduces neuropathic pain or the risk of developing neuropathic pain, treats a localized condition or reduces persistent postsurgical pain (PPP) in a subject.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### FIELD OF THE INVENTION

Tumescent solutions that contain a vasoconstrictor, a B vitamin that may impart a visible color to the solution, optionally another therapeutic agent or drug and a pharmaceutically acceptable carrier.

### BACKGROUND

Tumescent solutions contain a vasoconstrictor, e.g., epinephrine (1 mg/L) and a drug or therapeutic agent, wherein a tumescent concentration of the drug or therapeutic agent is simultaneously: 1) below a threshold for local, subcutaneous tissue toxicity, 2) above a threshold for positive local therapeutic effect, and 3) above a concentration safely achievable by intravenous (IV), intramuscular (IM) or oral (PO) delivery. For example, tumescent epinephrine lidocaine (TEL) includes epinephrine (1 mg/L) and lidocaine (1 gm/L) and is also known as tumescent local anesthesia or tumescent anesthesia. TEL is a 10-fold dilution of commercial FDA-approved 1% lidocaine with epinephrine 1:100,000 for infiltration local anesthesia.

For infiltrative local anesthesia in a 70 kg (154 pound) person, the FDA-approved 1% lidocaine with epinephrine has a maximal dosage and volume of 7 mg/kg and 50 ml. The FDA does not have any safety data to support these dosage restrictions. TEL, which is off-label, has a recommended maximal dosage and volume of 28 mg/kg and 2000 ml, with a 1:5,000,000 risk of mild lidocaine toxicity (Klein J A, Jeske D R. Estimated maximal safe dosage of tumescent lidocaine. Anesth Analg 2016; 122:1350-9). Thus, an off-label TEL solution consisting of 0.1% lidocaine and epinephrine 1:1,000,000 is safer, permits 4-times the mg/kg dosage and 40 times the total volume compared to on-label FDA-approved 1% lidocaine with epinephrine 1:100,000.

Subcutaneous infiltration of TEL for major surgical procedures in an operating room setting is increasingly common (Romero J, Bello J, Diaz J C, Grushko M, Velasco A, Zhang X, Briceno D, Gabr M, Purkayastha S, Alviz I, Polanco D, Della Rocca D, Krumerman A, Palma E, Lakkireddy D, Natale A, Di Biase L. Tumescent local anesthesia versus general anesthesia for subcutaneous implantable cardioverter-defibrillator implantation. Heart Rhythm. 2021, 18(8):1326-1335; Khater A, Mazy A, Gad M, Abd Eldayem O A, Hegazy M. Tumescent mastectomy: the current indications and operative tips and tricks. Breast Cancer. 2017; 9: 237-243; Hudson D A. The Value of Tumescent Infiltration in Bilateral Breast Reduction: Optimizing Vasoconstriction. Plast Reconstr Surg Glob Open. 2020; 8: e3050; Bashir M M, Sohail M, Wahab A, Umar Iqbal U, Qayyum R, NosheenJanf S. Outcomes of post burn flexion contracture release under tourniquet versus tumescent technique in children. Burns. 2018; 44: 678-682; Bitargil M, Kilic H E. Comparing local tumescent anesthesia and spinal anesthesia methods during and after endovenous radiofrequency ablation of great saphenous vein. Int Angiol. 2020; 39:461-466; Lalonde D, Martin A. Tumescent local anesthesia for hand surgery: improved results, cost effectiveness, and wide-awake patient satisfaction. Arch Plast Surg. 2014; 41:312-6; and Roerden, A; Neunhoeffer, F; Gotz, A; Hafner, HM; Kofler, L. Benefits, safety and side effects of tumescent local anesthesia in dermatologic surgery in infants. J Dtsch Dermatol Ges. 2021; 19:352-357; Ryota Koyama R, Yoshiaki M, Minagawa N, Shinohara T. The safety and feasibility of tumescent local anesthesia for inguinal hernia repair: A review of 273 consecutive patients. Asian J Surg. 2020; 43:700-701). A search of PubMed for “tumescent anesthesia” returns more than 662 articles. From a pharmacologic perspective, TEL is one of the world's safest drugs. When TEL is used appropriately as a subcutaneous injection it is extremely safe because of its very slow rate of systemic lidocaine absorption (Klein J A, Jeske D R. Estimated maximal safe dosage of tumescent lidocaine. Anesth Analg 2016; 122:1350-9; Klein J A. Tumescent technique for local anesthesia improves safety in large-volume liposuction. Plast Reconstr Surg. 1993; 92:1085-98; Makdisi J R, Kim D P, Klein P A, Klein J A. Tumescent contravenom: murine model for prehospital treatment of Naja naja neurotoxic snake envenomation. Int J Dermatol. 2018; 57:605-610; and Klein J A, Jeske D R. Estimated maximal safe dosage of tumescent lidocaine. Anesth Analg 2016; 122:1350-9).

An irony of tumescent solutions, e.g., TEL, is that their remarkable safety engenders complacency and the risk of human error. IV bags of saline and IV bags of TEL appear identical and are commonplace in surgical ORs. While an IV infusion of saline is safe, an IV infusion of TEL can be lethal. The vastly superior safety of TEL is tempered by the peril an inadvertent IV infusion of a TEL. If Murphy's Law is valid, then “If something can go wrong, it will.” The present patent application describes an unexpected and nonobvious solution to this TEL dilemma.

Vitamin B12 is useful in the treatment of pain and in promoting wound healing. B12 Combined with Lidocaine, Dexamethasone (corticosteroid) but not epinephrine, has been reported. The combination of dexamethasone-lidocaine-vitamin B12 mouth rinse may be moderately more effective than chlorhexidine mouthwash treating radiation mucositis (Li K, et al. “Radiation-induced mucositis: A retrospective study of dexamethasone-lidocaine-vitamin B12 mouth rinse versus compound chlorhexidine mouthwash in nasopharyngeal carcinoma. Heliyon. 2023; 9: e15955).

B12 and B complex vitamins, without epinephrine, have been used for treating nerve injury at a site of a peripheral nerve injury, using different drug delivery methods (Peritoneal B12 infusions (equivalent to intravenous infusions), Gan, L., et al., “Restorative effect and mechanism of mecobalamin on sciatic nerve crush injury in mice. Neural Regen Res. 2014; 9: 1979-1984; Ultrasound guided perineural B12 injections (Chen C H, et al., “Ultrasound-Guided Perineural Vitamin B12 Injection for Brachial Plexus Injury: A Preliminary Study”, Cell Transplant. 2023; 32: 09636897231167213); and implantation of B12 impregnated devices (Suzuki K, et al., “Electrospun nanofiber sheets incorporating methylcobalamin promote nerve regeneration and functional recovery in a rat sciatic nerve crush injury model”, Acta Biomater. 2017; 53: 250-259).

Treatment of Nerve Injury Pain with B12 and B complex vitamins, without Epinephrine, has been reported, for example in treating surgical nerve injury. B12 reduces pain associated with traumatic or inflammatory peripheral nerve injury including post-herpetic neuralgia, alcohol-related neuropathy and diabetic neuropathy (Julian T, et al., “B12 as a Treatment for Peripheral Neuropathic Pain: A Systematic Review”, Nutrients. 2020; 12:2221). Vitamin B12 may be helpful for treating pain with a low incidence of side effects (Buesing S, et al., “Vitamin B12 as a Treatment for Pain”, Pain Physician. 2019. 22: E45-E52; Alvarado A M and Navarro S A, “Complex B vitamins: Physiology and Therapeutic Effect on Pain”, Am J Pharmacolog Sci. 2016, 4:20-27; Wang, Z B, et al. “Thiamine, pyridoxine, cyanocobalamin and their combination inhibit thermal, but not mechanical hyperalgesia in rats with primary sensory neuron injury”, Pain. 2005; 114: 266-77).

B vitamins have been shown to promote wound healing. Treatment of experimental peripheral nerve injury with B vitamins: 1) promotes the transition of M1 neurotoxic proinflammatory macrophages to M2 antiinflammatory neuro-reparative macrophages, 2) reduces the extent of axon degeneration, and 3) accelerates the transition from non-myelin-forming to myelin-forming Schwann cells, which suggests B vitamins promote regeneration of injured nerves (Ehmedah A, et al. “Vitamin B Complex Treatment Attenuates Local Inflammation after Peripheral Nerve Injury”, Molecules. 2019, 24: 4615; and Ehmedah A, et al. “Effect of Vitamin B Complex Treatment on Macrophages to Schwann Cells Association during Neuroinflammation after Peripheral Nerve Injury”, Molecules. 2020 Nov. 19; 25(22): 5426).

B vitamins reduce neuroinflammation and suppress diabetic neuropathic pain and proinflammatory cytokines in diabetic neuropathic pain (He, D D, et al. “Systematic administration of B vitamins alleviates diabetic pain and inhibits associated expression of P2X3 and TRPV1 in dorsal root ganglion neurons and proinflammatory cytokines in spinal cord in rats. Pain Res Manag. 2020; 2020: 3740162).

B12 deficiency is associated poor wound healing. Supplemental B12 improves wound healing. There is a high incidence of B12 deficiency among diabetic peripheral neuropathy patients (Alvarez M, et al. “Vitamin B12 deficiency and diabetic neuropathy in patients taking metformin: a cross-sectional study”, Endocr Connect. 2019; 8: 1324-1329).

B12 induces peripheral nerve axonal and Schwann cell regrowth after rodent nerve crush injuries (Tamaddonfard E, et al. “Effect of vitamin B12 on functional recovery and histopathologic changes of tibial nerve-crushed rats. Drug Res. 2014, 64: 470-475; Erfanparast A, et al. ‘Systemic and local peripheral injections of vitamin B12 suppressed orofacial nociception induced by formalin in rats”, Drug Res. 2014; 64: 85-90; and Liao W C, et al., “Methylcobalamin facilitates collateral sprouting of donor axons and innervation of recipient muscle in end-to-side neurorrhaphy in rats”, PLoSONE 8: e763022013).

### SUMMARY

Some examples relate to a tumescent solution including:
a vasoconstrictor;
a B vitamin, and
a pharmaceutically acceptable carrier.

In some examples, the B vitamin is selected from the group consisting of B1 (Thiamine), B2 (Riboflavin), B3 (Niacin), B5 (Pantothenic acid), B6 (Pyridoxine), B7 (Biotin), B9 (Folic acid) and B12 (Cobalamin).

Some examples relate an intravenous (IV) fluid bag or container containing the tumescent solution.

In some examples, the B vitamin imparts a visible color to the tumescent solution is selected from the group consisting of Vitamin B12 and Vitamin B2.

In some examples, the tumescent solution further includes a local anesthetic.

In some examples, the local anesthetic is lidocaine.

In some examples, the B vitamin is vitamin B12.

In some examples, the concentration of vitamin B12 is in the range of 1-20 mg/L.

In some examples, the concentration of lidocaine is approximately 100 mg/L-2,500 mg/L of solution.

In some examples, the vasoconstrictor is epinephrine.

In some examples, the concentration of epinephrine is approximately 0.1 mg/L-2.5 mg/L.

In some examples, the tumescent solution further includes a drug or therapeutic agent dissolved in the tumescent solution, wherein a tumescent concentration of the drug or therapeutic agent is simultaneously:
1) below a threshold for local, subcutaneous tissue toxicity,
2) above a threshold for positive local therapeutic effect, and
3) above a concentration safely achievable by intravenous (IV), intramuscular (IM) or oral (PO) delivery.

In some examples, the drug or therapeutic agent is selected from the group consisting of:
(i) a chemotherapy agent,
(ii) an angiogenesis inhibitor,
(iii) an antiviral agent,
(iv) an antifungal agent,
(v) a biologic drug;
(vi) an antiprotozoal drug;
(vii) a selective serotonin reuptake inhibitor (SSRI);
(viii) an anti-seizure medication;
(ix) an antibiotic,
(x) an anti-inflammatory drug; and
(xi) an antivenom or contravenom.

Some examples relate to a method of subcutaneous delivery of a drug or therapeutic agent to a subject including subcutaneously administering to said subject a tumescent solution as disclosed herein.

In some examples, infiltration of the tumescent solution achieves both prolonged local drug or therapeutic agent concentration within a tumescent subcutaneous tissue as well as a prolonged slow constant systemic absorption of drugs from the tumescent tissue into a systemic circulation.

In some examples, a pharmacokinetic profile of the systemic absorption resembles a slow, constant, intravenous (IV) infusion.

In some examples, a subcutaneous concentration of the drug or therapeutic agent achieved is from about 1-100 times the maximum subcutaneous interstitial fluid concentration that can be achieved by conventional IV, IM or oral delivery of the drug or therapeutic agent.

In some examples, the drug or therapeutic agent reduces neuropathic pain or the risk of developing neuropathic pain.

In some examples, the neuropathic pain is selected from the group consisting of postherpetic neuralgia, trigeminal neuralgia, phantom limb pain, diabetic neuropathy, carpal tunnel syndrome, sciatica, degenerative disk disease, spinal cord injury, post-surgical pain and cancer.

Some examples relate to a method of subcutaneous delivery of a drug or a therapeutic agent to a subject to treat a localized condition, the method including: (a) obtaining a tumescent solution as disclosed herein, and (b) delivering a mass of the tumescent solution subcutaneously to a localized tissue in the subject, wherein the drug or the therapeutic agent treats the localized condition in the localized tissue, wherein a concentration of the drug or the therapeutic agent subcutaneously delivered exceeds a concentration of the drug or the therapeutic agent that can be safely achieved by intravenous delivery, and wherein an amount of the drug or the therapeutic agent located at an outer boundary of the mass of fluid and available for absorption is less than an amount of the drug or the therapeutic agent located within a central portion of the mass of fluid and virtually isolated from the systemic circulation by virtue of capillary vasoconstriction.

In some examples, the localized condition is selected from the group consisting of acute Herpes zoster pain, acute musculoskeletal pain, acute traumatic bone fractures, acute burns, acute blast injury, acute non-compressible hemorrhagic combat injury, postherpetic neuralgia, trigeminal neuralgia, phantom limb pain, diabetic neuropathy, carpal tunnel syndrome, sciatica, degenerative disk disease, spinal cord injury, post-surgical pain, cancer, a tumor, a surgical site infection, chronic post-operative pain, chronic alcohol consumption neuropathy, diabetic neuropathy, risk of deep vein thrombosis, risk of post-operative thromboembolism and a burn wound.

Some examples relate to a method of subcutaneous delivery of a drug or a therapeutic agent to a subject to treat a generalized condition, the method including:
(a) obtaining a tumescent solution as disclosed herein, and
(b) delivering a mass of the tumescent solution subcutaneously to a localized tissue in the subject,
wherein the drug or the therapeutic agent treats a generalized condition or a central nervous system process which affects tissue far beyond in the localized site of the injection.

In some examples, the method treats a condition selected from the group consisting of a cerebrovascular accident, multiple sclerosis, and a spinal cord injury.

Some examples relate to a method of reducing persistent postsurgical pain (PPP) in a subject including subcutaneously injecting into a surgical site of the subject a tumescent composition disclosed herein.

### DETAILED DESCRIPTION

Problems Associated with Tumescent Solutions Formulated in IV Bags

Other than a written label, there is no obvious means of easily and rapidly identifying an IV bag which contains a drug that might be toxic if given intravenously (e.g., 1 gm of lidocaine in a TEL solution). The use of warning-labels applied to the outside surface of IV bags containing a TEL solution is not an effective deterrent to an inadvertent IV infusion of TEL. Labels can be easily misread or misunderstood. Labels are ineffective if the personnel are not literate. Labels may be ineffective among persons who speak and read different languages. Labels may simply not be available when needed.

There is no FDA-approved color-indicator chemical or dye for aqueous solutions that is safe and nontoxic for subcutaneous, intravenous, or topical application.

There is no FDA-approved color indicator that is compatible with the chemical material used in the IV bags, IV tubing or the solutions commonly contained in IV bags such as a) balanced salt solutions (BSS) having a physiological pH and isotonic salt concentration, b) solutions of therapeutic drugs or chemicals intended for intravenous infusion, subcutaneous infiltration or topical irrigation of a body cavity or cutaneous surface.

There is a significant risk of toxicity associated with an inadvertent intravenous (IV) injection of an unmarked IV bag of a balanced salt solution that contains a drug or several drugs intended for subcutaneous infiltration or topical irrigation, but which might be toxic if infused intravenously.

It is a nonobvious possibility that anyone would intentionally put a drug in an IV bag if the drug might cause toxicity when infused intravenously.

The possible use of IV bags with specialized IV bag-like spike-ports and IV line-like connectors, which are not connectable to standard luer connectors used IV injections, is not an economically feasible solution to the problem of unintentional IV infusion of a tumescent solution. TEL solutions are used worldwide in many clinical settings including the office of individual healthcare providers, outpatient surgical facilities, and hospitals. The cost for purchasing and maintaining an entirely separate inventory of a drug delivery devices (IV bags and tubing) which are not compatible and not interconnectable with standard IV bags and tubing is not financially feasible and is not logistically practical.

Unexpected Inspiration for Using Non-Toxic Color Indicators, Such as Vitamin B12 and Vitamin B2

While reading about Herpes zoster therapies, Dr. Jeffrey Klein came across several obscure publications from the 1950's which reported that vitamin B12 has beneficial effects in treating shingles (HO J. “The treatment of herpes zoster with high doses of vitamin B12”. Z Haut Geschlechtskr. 1954; 17(12):378-81. (In German); Daste M. “Two cases of herpes zoster treated by vitamin B12 in doses of 1000 gammas”. Maroc Med. 1954; 33(351):789. (In French); Helle J, Ohela K. Treatment of herpes zoster neuralgia with massive doses of vitamin B12. Ann Med Exp Biol Fenn. 1955; 33(1-2):116-21; Matanic VI. “Ganglion-blocking agents and vitamin B12 in the treatment of bullous manifestations of herpes zoster”. Z Haut Geschlechtskr. 1958 Apr. 1; 24(7):192-7. (In German) and Jolles K E. Vitamin B12 in the treatment of herpes zoster. Med World. 1955; 83:433-5).

Further online research revealed a few additional publications supporting the benefits of B12 in treating the chronic pain of postherpetic neuralgia (Buesing S, Costa M, Schilling J M, Moeller-Bertram T. Vitamin B12 as a Treatment for Pain-Narrative Review. 2019; 22; E45-E52; Julian T, Syeed R, Glascow N, Angelopoulou E, Zis P. B12 as a Treatment for Peripheral Neuropathic Pain: A Systematic Review. Nutrients. 2020; 12: 2221; and Xu, G; Zhou, CS; Tang, WZ; Xu, J; Xu, G; Cheng, C; Wang, D; Kai Hua Ding, KH. Local Administration of Methylcobalamin for Subacute Ophthalmic Herpetic Neuralgia: A Randomized, Phase III Clinical Trial. 2020; 20:838-849).

Vitamin B12 produces a pink color and vitamin B2 produces a yellow color when dissolved in an aqueous solution. B12 and B2 promote regeneration of injured nerve fibers and wound healing (Mota I G, Das Neves RAM, Nascimento S S, Maciel B L L, Morais A H, Thais Souza Passos T S. 2021; Artificial Dyes: Health Risks and the Need for Revision of International Regulations, Food Reviews International).

Inspired by these reports, Dr. Klein made a conceptual extrapolation which led to a solution consisting of TELTA+vitamin B12. The result of this modification is TELTAB (Tumescent Epinephrine Lidocaine Triamcinolone Acyclovir B12 or B2). Subcutaneous injection of undiluted B12 is common. The concept of highly dilute tumescent B12 or B2 delivery had not previously been described.

When Klein placed a purchase order for his first vial of injectable B12, he had no idea that the solution would have a red/pink color. When 10 mg in 10 ml of B12 was added to 1,000 ml of TELTA (1:100 dilution of B12), the result was a TELTAB solution with a distinctive pink color. It had a rather pleasing aesthetic appeal. He thought the novel pink color would be memorable and an interesting topic of conversation. It did not occur to him that B12 had any practical advantage other than a purely therapeutic benefit. The next conceptual step was the idea that the pink color of TELTAB might be a clever marketing gimmick, which could distinguish TELTAB from all other bags of saline. Vitamin B12 (for example 10 mg/10 ml) imparts a distinctive pink color to a TEL solution. This colored solution provides a highly visible warning that the IV bag contains a drug that should “NOT be given intravenously.” Although the nutritional function of Vitamin B12-for-injection is not stable when exposed to light, its pink pigment does not appear to fade over time.

Jeffrey Klein and his son Bram Klein had several discussions concerned with the possible advantages of using the pink appearance of TELTAB as a marketing strategy to distinguish TELTAB from all other IV bags of saline. It was during such a discussion that they both realized a more generalized advantage of adding B12 to any IV bag containing TEL. Vitamin B12 can distinguish any IV bag containing TEL from all other IV bags that do not contain TEL. It was their “Aha!” moment. This advantage of using B12 or B2 was an unexpected and nonobvious answer to the question of how to reduce the risk of an inadvertent IV infusion of TEL. The recognition that B12 and B2 are non-toxic, FDA approved drugs that can act as color-indicators that can distinguish an IV bag containing TEL from all other IV bags was an epiphany.

An intensive online search of the literature found no evidence that anyone had ever added a colored dye to an IV bag of saline simply to give the solution a distinctive color. There are no examples of prior use of non-toxic color-indicator dyes specifically designated for aqueous solutions contained in IV bags. The search found that there are only nine types of FDA-approved food-colors, and each of these food-colors has some toxic effect that strictly limits its daily consumption. Food colors are the only colored dyes that are FDA approved for human use. Thus, there has heretofore been a lack of known non-toxic compounds that can be safely used in the context of solutions administered to subjects.

B12 and B2 do not have FDA approval for use as a color-indicator for solutions intended for parenteral delivery. Therefore, the use of B12 or B2 in an IV bag of saline is considered “off-label.” Klein is currently pursuing an FDA investigational new drug (IND) application for permission to conduct a randomized clinical trial (RCT) of tumescent delivery of subcutaneous B12 and B2.

The inspiration for using B12 and B2 as a non-verbal warning against an IV injection of a TEL solution is a nonobvious idea. The cognitive path that led to the realization that B12 could and should be used as a colored safety indicator for TEL was obscure and unexpected.

Klein took the first step along this path while studying and perfecting the composition for TELTA (tumescent epinephrine lidocaine triamcinolone acyclovir), which is his novel treatment for the most severely painful cases of Herpes zoster (shingles). For several years he has had remarkable success in treating patients with the most severe acute Herpes zoster pain using TEL+triamcinolone+acyclovir=TELTA. TELTA can consist of a 1 liter intravenous (IV) bag of normal saline (NS) together with lidocaine (700 mg/L), epinephrine (0.7 mg/L), triamcinolone (40 mg/L), and acyclovir (1 gm/L)=TELTA. See U.S. Pat. No. 10,493,024 B2.

This patent was issued because the tumescent technique, which permits subcutaneous injection of dilute acyclovir, is novel and entirely unexpected. The FDA package insert for “acyclovir for injection” states that acyclovir “should not be injected subcutaneously.” Consequently, no one had ever considered the use of subcutaneous injections of acyclovir to treat Herpes simplex. Klein discovered that with sufficient dilution, subcutaneous tumescent acyclovir is both safe and highly effective. U.S. Pat. No. 10,493,024 B2 provides an example of the potential for tumescent drug delivery of multiple drugs in a single tumescent solution.

Unexpected Advantages of Formulating Tumescent Solutions with Vitamin B12 and/or Vitamin B2

Color-Indicators (benign and nontoxic) for therapeutic solutions intended for subcutaneous infiltration, irrigation of wounds or body cavities or intravenous infusion is a logistically practical and economically feasible resolution for the problems described above.

Color-indicators (e.g., B12 and/or B2) can readily identify solutions in an IV bag that must never be infused intravenously and are only intended for subcutaneous injection, topical application, or intracavitary irrigation.

Color-indicators (e.g., B12 and/or B2) provide a visible and non-verbal indication that the contents of a solution in an IV bag are not the typical or expected saline solutions and are not intended for routine IV delivery.

Color-indicators, such as B12 and/or B2, are safe because they have virtually no drug interactions with other therapeutic drugs which may be included in bags tumescent solutions.

Color-indicators might be chemically inert or therapeutically beneficial such as B12 and/or B2.

A different set of color indicators may likewise improve patient safety when used to specifically identify a solution contained in an intravenous bag which is intended for IV infusion and not intended for subcutaneous infiltration.

B Vitamin Compositions

Disclosed herein are compositions that include either:
Tumescent Epinephrine B complex vitamin(s) (e.g., B12) (TEB), a basic composition, or
Tumescent Epinephrine Lidocaine B complex vitamin(s) (TELB), a clinically important composition,
where B represents B12 or any of the 8 vitamin B complex vitamins dissolved in a pharmaceutically acceptable excipient, such as normal saline (NS) or a lactated Ringer's (LR) solution.

Also disclosed herein are excipient solutions for tumescent drug delivery, including TELB for the purpose of:
1) A local anesthetic solution that is uniquely capable of providing pure sensory random area regional anesthesia,
2) An excipient (carrier solution) for tumescent drug delivery of any appropriate FDA-approved injectable drug (D), or
3) providing local capillary vasoconstriction.

The B Vitamins include: B1 (Thiamine), B2 (Riboflavin), B3 (Niacin), B5 (Pantothenic acid), B6 (Pyridoxine), B7 (Biotin), B9 (Folic acid) and B12 (Cobalamin). There are several equivalent therapeutic formulations for vitamin B12, including Methylcobalamin, Hydroxycobalamin and Cyanocobalamin.

B12 is a preferred vitamin in the compositions disclosed herein. In some embodiments, B1, B6, and B12 are B vitamins useful for wound healing. B1, Thiamine; B6, Pyridoxine; and B12, Cobalamin are neurotrophic and are useful with respect to the health of peripheral nerves and healing of surgically injured nerves.

Any of the individual B vitamins, as well as any combination thereof, can be included in the compositions disclosed herein.

Subcutaneous TEL solution containing B12 provides exceptionally high and unmatched local B12 bioavailability. Vitamins B1, B6, and B12 improve healing and axon repair following peripheral nerve injury (PNI), including traumatic PNI caused by a surgical incision.

To the extent that B complex vitamins improve and accelerate postsurgical wound healing, TELB can be expected to achieve optimal B vitamin wound-healing effects.

PNI recovery is facilitated by vitamin B12, as well as by vitamin B1 and vitamin B6. The precision subcutaneous tumescent infiltration of B12, as well as B1 or B6, at a site of a proposed surgical incision will: 1) decrease proinflammatory M1 tissue resident macrophages and increase anti-inflammatory M2 perineural macrophages following PNI, 2) accelerate Schwann cell regrowth, 3) accelerate wound axon repair following PNI, and significantly contribute to the prevention of persistent postsurgical pain (PPP). Therapeutic doses of vitamin B12 are generally >100 μg/dose, but may be anywhere in the range of 50-10000 μg/dose, including dosages of 50, 100, 150, 200, 250, 300, 350, 400, 450, 500, 550, 600, 650, 700, 750, 800, 850, 900, 950, 1000, 1100, 1150, 1200, 1250, 1300, 1350, 1400, 1450, 1500, 1550, 1600, 1650, 1700, 1750, 1800, 1850, 1900, 1950, 2000, 2250, 2500, 2750, 3000, 3250, 3500, 3750, 4000, 4250, 4500, 4750, 5000, 5500, 6000, 6500, 7000, 7500, 8000, 8500, 9000, 9500 and 10000 μg/dose. The concentration of vitamin B12 used in a tumescent solution may be in the range of 1 mg/L-20 mg/L, including intermediate values of 2 mg/L, 3 mg/L, 4 mg/L, 5 mg/L, 6 mg/L, 7 mg/L, 8 mg/L, 9 mg/L, 10 mg/L, 11 mg/L, 12 mg/L, 13 mg/L, 14 mg/L, 15 mg/L, 16 mg/L, 17 mg/L, 18 mg/L, and 19 mg/L.

Tumescent Definitions

Tumescent is an adjective. Tumescent tissue is tissue that is swollen and firm, derived from the Latin verb tumescere: to swell up and become firm.

Tumescence is a noun. A tissue possesses tumescence, has tumescence, or displays tumescence when it is swollen and firm. Tumescence is required to achieve optimal widespread dispersion of a dilute epinephrine solution throughout targeted subcutaneous tissue.

Detumescent is an adjective. Detumescent tissue is tissue that has become less swollen and less firm.

Detumescence is a noun. A tissue achieves detumescence after it has been allowed to become less swollen and less firm.

TE (tumescent epinephrine) solution consists of dilute epinephrine dissolved in normal saline or lactated Ringer's (LR) solution at concentrations of 0.1 mg/L-2.5 mg/L. A subcutaneous injection of a relatively large volume of a relatively dilute solution of tumescent epinephrine induces extensive widespread local capillary vasoconstriction. TE, at 1 mg/500 ml, is an effective contravenom when injected immediately after a snakebite envenomation at the site of a snakebite (Makdisi J R, et al. “Tumescent contravenom: murine model for prehospital treatment of Naja naja neurotoxic snake envenomation”, Int J Dermatol 2018; 57:605-610).

TEB consists of Tumescent Epinephrine and vitamin B12 (with or without other B complex vitamins).

TEL (Tumescent Epinephrine Lidocaine) solution consists of dilute epinephrine at concentrations of 0.1 mg/L-2.5 mg/L and dilute lidocaine at concentrations 100 mg/L-2,500 mg/L. TEL provides pure sensory random regional anesthesia. For most clinical applications, the concentration of epinephrine and lidocaine in a TEL are [≤1 gm/L] and [≤1 mg/L], respectively.

TELB (Tumescent Epinephrine Lidocaine vitamin B12), with or without other B Complex vitamins, consists of TEL plus B12 (approximately 10 mg/L).

Tumescent Delivery (TD) of a drug (D) is defined as the subcutaneous injection of a relatively large volume of a relatively dilute solution of an injectable water-soluble drug D dissolved in a TEB or TELB or TEL solution. TD is a novel mode of drug delivery which has unsurpassed pharmacokinetic and pharmacodynamic advantages compared to traditional IV, IM and PO modes drug delivery. TEL, TEB and TELB are highly effective excipient (carrier) solutions for subcutaneous delivery of drug D. TD of drug D assures a peak interstitial concentration of D that is precisely equal the concentrations of drug D within the TD solution. The bioavailability of drug D within the targeted interstitial tissue is typically at least 10 to 100 times that which can be achieved by IV delivery of drug D.

Pharmacology is the study of a drug's molecular, biochemical, and physiologic effects or actions.

Lidocaine: Lidocaine is an amide-type local anesthetic. Not only is lidocaine probably the safest and most effective of all local anesthetic drugs. In fact, lidocaine is among the safest and most effective of all drugs. Importantly, lidocaine has a surprising number of valuable therapeutic qualities, including:

Nociceptor inhibition: Lidocaine in TEL prevents noxious conditions from initiating of sensory nerve impulses which would otherwise be perceived as pain (Roberson D P, et al. “Targeting of sodium channel blockers into nociceptors to produce long-duration analgesia: a systematic study and review”, Br J Pharmacology. 2011; 164 48-58).

Antiinflammatory (lidocaine in TEL has surprising antiinflammatory effects comparable to betamethasone). Injections of lidocaine or betamethasone have comparable antiinflammatory effects in alleviating inflammation in carpal tunnel syndrome mononeuropathy (Dernek B, et al. “Comparison of the efficacy of lidocaine and betamethasone dipropionate in carpal tunnel syndrome injection”, Clinical Trial J Back Musculoskelet Rehabil. 2017; 30:435-440).

Bactericidal: Lidocaine in TEL is bactericidal and thus reduces risk of surgical site infections (Razavi B M and Bazzaz B S F. Review—A review and new insights to antimicrobial action of local anesthetics. Eur J Clin Microbiol Infect Dis. 2019; 38: 991-1002).

Antiplatelet: Lidocaine in TEL inhibits platelet activation and reduces risk of perioperative venous thromboembolism (VTE), Norman N C, et al. “Pulmonary Embolism after Liposuction Totally by Tumescent Local Anesthesia in a Patient with Large Uterine Fibroids”, Case Report. Plast Reconstr Surg Glob Open. 2023 Mar. 13; 11(3): e4876).

Intravenous (IV) Lidocaine: The magnitude of therapeutic (antiinflammatory) serum lidocaine concentrations are similar following either IV delivery or subcutaneous TEL infiltration. However, TEL is associated with more prolonged therapeutic lidocaine concentrations (Klein J A and Jeske D R. Estimated maximal safe dosage of tumescent lidocaine. Anesth Analg. 2016; 122:1350-1359).

Lidocaine effects the peripheral nervous system (PNS) and the central nervous system (CNS), via silencing ectopic discharges, suppression of inflammatory processes, and modulation of inhibitory and excitatory neurotransmission (2019 Hermanns H, et al. “Molecular mechanisms of action of systemic lidocaine in acute and chronic pain: a narrative review”, Br J Anaesth. 2019; 123: 335-349).

IV lidocaine is effective in pain control for neuropathic pain in the immediate post-infusion period. IV lidocaine does not have a long-lasting, persistent effect (2019 Zhu B, et al. “Intra-Venous Lidocaine to Relieve Neuropathic Pain—A Systematic Review and Meta-Analysis”. Front Neurol. 2019; 10: 954).

IV lidocaine infusions are used for postoperative analgesia in clinical practice. The postoperative analgesic and anti-hyperalgesic effects of IV lidocaine were confirmed in later studies. Lidocaine has anti-nociceptive, anti-hyperalgesic and anti-inflammatory effects (Foo I, et al. “The use of intravenous lidocaine for postoperative pain and recovery-international consensus statement on efficacy and safety”, Anaesthesia 2021, 76: 238-250).

Detumescent is an adjective that describes tumescent tissue which has becomes less swollen and less firm. Allowing sufficient time for adequate detumescence (noun) is essential for optimal surgical vasoconstriction and surgical hemostasis anesthesia. Detumescence begins immediately after tumescent infiltration has ceased. Hydrostatic pressure gradient within tumescent tissue causes fluid to spread outwardly by bulk flow through the interstitial gel substance. Depending on the volume of tumescent fluid, an adequate duration of pre-incision detumescence can be 15 to 120 minutes. With insufficient detumescence, the incised tissue will be excessively “water-logged” and awkward to manipulate. Any deliberate effort to decrease the time required for adequate detumescence by reducing the volume of tumescent fluid will yield inadequate hemostasis and suboptimal postsurgical analgesia. For example, for the excision of a 10 mm diameter melanoma with 10 mm surgical margins:
adequate anesthesia requires 60 ml to 90 ml of tumescence,
good hemostasis requires 160 ml to 200 ml provides, and
pain-free postsurgical analgesia requires 275 to 550 ml.

The large volume of tumescence provides an effective, long-lasting subcutaneous reservoir of tumescent fluid.

Pharmacodynamics (PD) (derived from Greek: pharmakon=drug, and dynamikos=power) is the branch of pharmacology concerned with a drug's effects as a function of its tissue concentration (ml=mm 3) at its site of action. PD is a 3-dimensional (mm 3) concept. Local anesthesia is commonly conceived of as a 3-dimensional process, wherein the total volume directly determines the clinical effect.

Pharmacokinetics (PK) (derived from Greek: pharmakon=drug, and kinein (verb)=to move) is the branch of pharmacology concerned with a drug's tissue concentration as a function of time. PK is a 4-dimensional (mm³, time) concept.

Duration of Detumescence is the length of time between completion of tumescent infiltration and the initial surgical incision. For surgical applications of TEL, the duration of detumescence is an essential determinant of TEL efficacy. Time is of the essence.

TELA (tumescent epinephrine lidocaine anesthesia) designates the use of TEL for surgical local anesthesia. TELA is a 4-dimensional process.

TELE (tumescent epinephrine lidocaine excipient) designates the use of TEL as the excipient solution for subcutaneous and intradermal tumescent delivery (TD) of one or more injectable drugs.

Ambit is defined as the extent, bounds, scope of something. Ambit is the root of “ambitious.”

Ambit of inflammation of a painful event, or condition, is the extent (surface area×depth=volume) of tissue within the receptive field of stimulated nociceptors. Tissues adjacent to a surgical incision site or a dermatome affected by H zoster are examples of the ambit of inflammation. Stimulated nociceptors plausibly lower the activation threshold of adjacent nociceptors, thereby further enlarging the ambit of inflammation.

Ambit of TDD the extent (volume=surface area×depth) of tissue (subcutaneous and intradermal) that has been infiltrated with a TDD solution.

Bioavailability is a mathematical concept for comparing efficacy of two different modes of drug delivery. An understanding of bioavailability is necessary to fully appreciate the superior safety and efficacy of the Tumescent Drug Delivery (TDD) of drug D compared to the safety and efficacy IV, IM, and PO delivery of drug D, when the target of drug D is localized cutaneous tissue. For example, when the targeted tissue is a dermatome affected by acute Herpes zoster, TDD of acyclovir is far more effective than IV or PO delivery of acyclovir.

AUC=Area Under the Curve is defined by the integral (area under the curve) of a drug's concentration in a specified tissue as a function of time (t), where t ranges from time (t=0) when the drug is first administered, to the time (t=oo) when the drug is no longer detectable in the tissue. AUC is proportional to the total (mg) dose of a drug that enters the specified tissue.

TEB and TELB provide surprising and unexpected positive results in the treatment of peripheral nerve injury (PNI).

Tumescent B12 Improves Postherpetic Neuralgia

Herpes zoster (shingles) is a painful viral infection (varicella virus) that causes significant peripheral nerve injury (PNI) within to a single unilateral dermatome. When the intense pain of H. zoster (shingles) persists for months (≥3) or years, it is known as postherpetic neuralgia (PHN). Currently there is no recognized effective treatment for PHN.

Tumescent Epinephrine, Lidocaine, vitamin B12, Acyclovir, Triamcinolone (TELB [AT]) provides long term improvement of PHN. The inventor has treated more than a hundred patients with severely painful H. zoster with numerical pain scale scores ≥7/10. These patients were treated with a combination of drugs in a solution consisting of Tumescent Epinephrine, Lidocaine, Acyclovir, Triamcinolone or TEL[AT]. Subcutaneous TEL[AT] provides instantaneous relief of H. zoster pain for 12 to 24 hours. Compared to the standard treatment of acute H. zoster with oral valacyclovir, daily subcutaneous injections of TEL[AT] within the effected dermatome reduces the duration of shingles pain and shingles rash by half.

The first hint of the clinical benefits of tumescent delivery of vitamin B12 was seen following the addition of B12 (10 mg/L) to a TEL[AT] solution. The augmented TELB [AT] solution has proven to be superior to TEL[AT] in several respects.

1) While TEL[AT] and TELB [AT] are equally effective in rapidly arresting all progression of the Herpes zoster vesicular dermatitis, TELB [AT] noticeably shortens the duration of the H. zoster pain by days. Significantly, the use of B12 has reduced the incidence of PHN.

2) Even more surprising, in every patient who has presented with established PHN, TELB [AT] treatment has permanently eliminated approximately 75% of the skin surface area affected by PHN pain.

3) The use of TELB [AT] for PHN has revealed the existence of two clinically distinct areas of painful skin within the affected dermatome. “Hotspot pain” is the area within the dermatome where the zoster pain is clearly the most intense. Virtually every shingles patient can identify a hotspot. “Elsewhere pain” is zoster pain that affects areas within the dermatome other than the hotspot.

In acute H. zoster, elsewhere pain is resolves more quickly than hotspot pain. Among patients with PHN, TELB [AT] usually permanently eliminates the elsewhere pain.

TELB [AT] treatment results of PHN hotspot pain vary. A single treatment typically provides noticeable pain relief for one to seven days. Repeated treatments every two weeks have provided gratifying long-term improvement with respect to average daily NPS scores, but pain level tend vary from day to day.

Epinephrine in combination with B12 has not been reported. There are published reports of the combination of B12 and Lidocaine without epinephrine. Intralesional B12+Lidocaine injections without epinephrine have been reported (Xu G, et al. “Pain Fluctuations of Women with Subacute Herpetic Neuralgia During Local Methylcobalamin in Combination with Lidocaine Treatment: A Single-Blinded Randomized Controlled Trial”, J Pain Res. 2023; 16: 1267-1284).

B12 (1000-1500 μg, plus 2.0% lidocaine (20-30 mg, in 4 to 6 ml local injection, 0.3-0.5 mL increments into the most painful area. There was no epinephrine in the solution (Xu G, et al. “Local Administration of Methylcobalamin for Subacute Ophthalmic Herpetic Neuralgia: A Randomized, Phase III Clinical Trial”, Pain Pract. 2020; 20: 838-849).

Intralesional B12 with lidocaine is superior to systemic delivery of B12. Local injections of B12 with lidocaine are significantly better than Local lidocaine injections with systemic B12 (either IM B12 or PO B12), Xii. G, et al. “Local Injection of Methylcobalamin Combined with Lidocaine for Acute Herpetic Neuralgia”, Pain Med. 2016; 17: 572-581.

TDELB[C] Surprisingly Eliminates all Postsurgical Incision-Site Pain

Tume sc ent-Detume scent Epinephrine, Lidocaine, vitamin B12, and Clindamycin or Cefazolin TDELB [C] completely eliminate postsurgical incision-site pain. This is entirely an unexpected result. This dramatic elimination of pain is totally unexpected by anyone familiar with the art of surgical anesthesia, including surgeons, anesthesiologists, pain management specialists and the editors of their respective scientific journals.

Persistent postsurgical pain (PPP) is a major problem following many surgeries accomplished under general anesthesia, whenever pre-incision TEL is not used.

Incision-site PPP and RP are local phenomenon, affecting well defined, extensive peri-incisional tissue. Yet anesthesiologist, surgeons, and pain management specialists uniformly have generally accepted a theory that attributes persistent incision-site pain to neuropathic changes in sensory nerves proximal to the injury, the dorsal root ganglia and “central sensitization” within the CNS. Consequently, current therapeutic techniques for treating PPP and RP employ standard IV, IM, and PO modes of systemic drug delivery.

Physician who are “skilled in the art of pain management” completely unaware of the tumescent technique for drug delivery of injectable drugs. Although dermatologists and plastic surgeons are generally familiar with tumescent epinephrine lidocaine (TEL) for liposuction surgery, virtually no physicians know about the benefits of tumescent delivery (TD) of vitamin B12.

Tumescent Detumescent Epinephrine Lidocaine Vitamin B12 (TDELB)

Current anesthesiology research is exclusively focused on treating, rather than preventing PPP. Anesthesiologists are unaware of the TDELB (tumescent-detumescent) epinephrine lidocaine B12 technique for local anesthesia. TDELB requires both: 1) preincisional subcutaneous infiltration of large volumes of TEL solution and 2) at least one to three hours of pre-incision detumescence.

Anesthesiologists and surgeons have never thought of TDELB as a means of reducing postsurgical pain because TDELB is incompatible with exigencies of standard operating room workflow patterns because painless subcutaneous infiltration of TDELB: 1) is time consuming, 2) the duration of time required for adequate detumescence, requires that the infiltration of TDELB be completed anywhere from one to two hours before surgery, 3) TDELB is off-label and not yet FDA approved, 4) there is no CPT code for TDELB infiltration and therefore TDELB is not covered by health insurance companies. Thus, economic considerations of operating room anesthesiology preclude the routine use of TDELB.

In the inventor's clinical experience, for large dermatologic excisions, the B12 component of TDELB also reduces the postsurgical incision-site inflammation and numbness. B12 is known to be anti-inflammatory and to promote healing of traumatized peripheral sensory function.

It is known that acute peripheral nerve injury can be effectively treated with vitamin B12. Several modes of B12 delivery by injection have been described. None of these techniques inject a solution of B12 combined with epinephrine. All of these drug delivery techniques use small volumes (a few ml) of commercial B12 solutions/concentrations with little or no dilution.

When a solution of B12 and dilute epinephrine is injected into subcutaneous tissue, the epinephrine induces local capillary vasoconstriction which delays the systemic absorption of the B12 and prolongs and augments the local effects of B12 within the injected tissue. None of the techniques for B12 injection described in the literature have considered the tissue concentration of B12 as a function of time (pharmacokinetics). None of the techniques for B12 injection described in the literature have added epinephrine in the injected solution in order to induce local capillary vasoconstriction and delay systemic absorption of the B12.

When a given amount of B12 is diluted in a large volume of NS or LR, and the entire volume is injected into the targeted tissue, then a greater proportion of the targeted tissue will be directly exposed to B12. None of the techniques for injecting B12 use large volumes of highly dilute B12 in order to expose large volumes of subcutaneous tissue to the B12.

None of the published techniques for targeted B12 injections consider the biologic effect of B12 as a function of the B12 concentration (pharmacodynamic). In fact, the concentration of B12 in tumescent solution may be in the range of 1 μg/ml-20 μg/ml, which equals 1,000 ng/ml-20,000 ng/ml. Serum and tissue concentrations of B12 in a healthy individual are ≤1 ng/ml. From a pharmacodynamic perspective, it is clear that the concentration of B12 in a tumescent solution is not deficient.

It is not obvious or well appreciated that, in a busy operating room, a 1 liter IV bag containing TEL might be mistaken for an IV bag of NS or LR solution. Large volumes of dilute tumescent epinephrine lidocaine (TEL) are increasingly utilized in operating rooms during surgeries by plastic surgeons, orthopedic surgeons, and burn surgeons. IV bags of TEL are routinely prepared in common 1 liter IV bags containing NS or LR solutions. In many cases, IV bags of TEL do not have labels which clearly distinguish TEL IV bags from any other IV bags in a busy operating room. Even when an IV bag of TEL is well labelled, it possible that a hurried staff in the hurried setting of operating room might confuse a TEL IV bag with a normal IV bag and inadvertently connect the TEL IV bag to an intravenous line and infuse the TEL solution directly into a patient's blood stream. A rapid intravenous infusion of 1 gm of lidocaine and 1 mg of epinephrine can be fatal.

Based on facts gleaned from the current literature: 1) It is unexpected that there is a distinct clinical advantage to combining B12 and epinephrine when a subcutaneous injection of B12 is required; 2) It is unexpected that there are significant therapeutic advantages to combining B12 and epinephrine in highly dilute solutions when B12 is intended for subcutaneous injection; 3) It is unexpected that a highly dilute tumescent solution of B12 together with epinephrine when used as an excipient solution for a subcutaneous or intradermal injection of a local anesthetic prior to a routine surgical incision, with or without general anesthesia, can hasten the repair of any surgically induced peripheral nerve injury; and 4) It is unexpected that the addition of B12 (10 mg/10 ml) to a 1 liter bag of TEL can prevent the inadvertent intravenous infusion of the TEL. There is no FDA approved colored chemical that is entirely safe when added to an IV bag and injected into a patient. The addition of 10 ml of B12 (1 mg/ml) into a 1 liter bag of NS or LR imparts a distinct pink-red color to the solution.

TEL[AT] contains Epinephrine, Lidocaine, Acyclovir, and Triamcinolone is used to treat acute Herpes zoster (shingles). TEL[BAT], with B12 is clinically superior to TEL[AT] without B12, with respect to elimination of severe Herpes zoster with one to five daily treatments among patients with 8/10 zoster pain.

Unexpected Prevention of Persistent Postsurgical Pain (PPP)

There are no generally recognized effective means of PPP prevention or cure. Among anyone familiar with the art of pain management, it is surprising, not anticipated, and unexpected that TEL or TELB:

1) Prevents all acute incision-site pain and surgical site bleeding for the largest dermatologic surgical excision, including large volume tumescent liposuction totally by local anesthesia. (Klein J A. Tumescent technique for local anesthesia improves safety in large volume liposuction. Plast Reconstr Surg 1993; 92:1085-1098).

2) Prevents 100% of perioperative opioid requirements associated with incision-site pain. (Klein J A. Tumescent technique for local anesthesia improves safety in large volume liposuction. Plast Reconstr Surg 1993; 92:1085-1098).

3) Greatly reduces the risk of postsurgical venous thromboembolism (VTE) for large cutaneous excision and for large volume liposuction. (Norman N C, et al. “Pulmonary Embolism after Liposuction Totally by Tumescent Local Anesthesia in a Patient with Large Uterine Fibroids. Case Report”, Plast Reconstr Surg Glob Open. 2023 Mar. 13; 11(3): e4876).

4) Greatly reduces the risk of surgical site infections (SSI) in large volume liposuction. (Klein J A and Langman L J. “Tumescent anesthesia antibiotic delivery: pharmacokinetics of subcutaneous cefazolin and metronidazole in a tumescent solution for prevention of surgical infections and biofilms”, Plast Reconstr Surg Glob Open 2017; 5: e1351).

PPP—TELB Eliminates Persistent Postsurgical Pain (PPP)

Anesthesiologists are unaware of effective treatments for severe persistent postsurgical pain (PPP) or severe rebound pain (RP) following nerve block anesthesia. PPP and RP are distressingly common occurrences.

Preemptive, preincisional widespread subcutaneous infiltration in a tumescent epinephrine, lidocaine, B12 solution (TELB [clindamycin] and TELB [cefazolin]) prevents nearly 100% the occurrence acute post-surgical pain in more than 80% patients undergoing large skin incisions.

Sufficiently large volumes of preincisional (TELB [Cefazolin] or TELB [clindamycin] together with sufficient detumescence, prevents the experience of acute postsurgical incision-site pain. If there is no acute postsurgical incision-site pain, there cannot be persistent postsurgical incision-site pain.

In my personal clinical experience, the preemptive subcutaneous skillful injection of a sufficient volume of a dilute tumescent solution containing vitamin B12 [10 mg/L], epinephrin [1 mg/L], lidocaine [1 gm/L], and an injectable antibiotic, such as clindamycin [900 mg/L] or cefazolin [1 gm/L], followed by ≥1 hour pre-incision pause to allow sufficient time for detumescence, unfailingly eliminates two of the most vexing and painful postsurgical conditions: 1) persistent postsurgical pain (PPP), and 2) rebound pain (RP) after nerve block anesthesia.

PPP is a vexing problem that adversely affects the health and quality of life (QOL) in 2% to 10% of patients following many common surgical procedures, especially mastectomy, thoracotomy, sternotomy, amputation and inguinal or abdominal surgeries.

Persistent Postsurgical Pain (PPP) is Prevented by TELB

For treating acute Herpes zoster, subcutaneous infiltration of tumescent epinephrine, lidocaine and B12, provides wide-ambit pure sensory regional anesthesia and immediately provides complete pain relief for more than 24 hours.

For treating surgical incision sites, enduring elimination of acute postsurgical incision-site pain by subcutaneous infiltration of tumescent epinephrine, lidocaine and B12, immediately provides continuous and permanent wide-ambit pure sensory regional anesthesia.

Persistent postsurgical pain (PPP) and chronic postsurgical neuropathic pain are a significant risk associated with surgical procedures routinely accomplished under general anesthesia (GA) or nerve block anesthesia (NBA), (Thapa P and Euasobhon P. “Chronic postsurgical pain: current evidence for prevention and management”, Korean J Pain. 2018; 31: 155-173).

Persistent postsurgical pain (PPP) is significant in 10-50% of common surgical procedures and severe in 2-10% of cases. PPP is especially problematic groin hernia repair, breast and thoracic surgery, leg amputation, and coronary artery bypass surgery (Kehlet H, Jensen T S, Woolf C J. Persistent postsurgical pain: risk factors and prevention” Lancet. 2006; 367: 1618-25).

Since chronic pain can be severe in about 2-10% of these patients, persistent postsurgical pain (PPP) represents a major, largely unrecognized clinical problem. To the best of our knowledge, published reviews and case-series accounts of PPP prevention or PPP treatment have not recommended the use of infiltration local anesthesia.

In contrast, dermatologic surgeons uniformly rely on infiltration local anesthesia for virtually every surgical procedure. To the best of our knowledge, PPP has not been reported following large dermatologic surgical procedures, including large incisions, extensive excisions, and large volume liposuction procedures.

This suggests that the use of large volume tumescent-detumescent epinephrine lidocaine may significantly reduce or eliminate the incidence of PPP in general surgery.

Common Application for B12 Therapy

The biological effect of B12 is to act as a cofactor in enzymatic reactions that play a role in the synthesis of DNA, myelin, and fatty acids, which are vital for cell division and growth. These are generally regarded as systemic effects. In clinical medicine, the therapeutic use of vitamin B12 is almost exclusively intended for treating or preventing the effects of systemic B12 deficiency.

Vitamin B12 deficiency may induce macrocytosis, peripheral neuropathy, ataxia, dizziness, cognitive disturbances, depression, and delirium.

FDA approved indications include the following:
Pernicious anemia: Intrinsic factor of Castle deficiency due to autoantibody formation against parietal cells of the stomach, which results in decreased absorption of vitamin B12 through ileum.
Malabsorption: Impairment of vitamin B12 absorption.
Atrophic gastritis: Intrinsic factor level decreases and leads to reduced absorption of vitamin B12.
Chronic acid-reducing medication use: Chronic reduction of acid secretion decreases the release of bound vitamin B12 from the protein of food particles, so R factor is unable to bind, and ultimately vitamin B12 absorption is reduced.
Long-term metformin use: May correlate with reduced intestinal mobility or intestinal bacterial overgrowth that competes with vitamin B12 and decreases its absorption.
Total or partial gastrectomy: It reduces the level of the intrinsic factor of Castle needed for vitamin B12 absorption, so decreased intrinsic factor leads to decreased vitamin B12 absorption.
Small bowel bacteria overgrowth: Vitamin B12 is overused by bacteria leading to B12 deficiency.
Diphyllobothrium latum infection: Competes with vitamin B12 for absorption through the intestine.
Pancreatic insufficiency
Helicobacter pylori infection
Dietary deficiency of vitamin B12

Non-FDA approved indications:
Cyanide poisoning
Smoke inhalation
Surgery-associated vasoplegia
Folic acid deficiency

TE, TEB, TEL, and TELB are Related Basic Tumescent Compositions

TE (tumescent epinephrine) represents a tumescent solution containing epinephrine in NS or RL. TE can function as an excipient for tumescent drug delivery, but it is not capable of masking the pain-upon-injection that some drugs cause.

TE provides tumescent capillary vasoconstriction. Targeted subcutaneous and intramuscular injection of a TE soon after a venomous snakebite produces widespread localized capillary vasoconstriction which delays the systemic absorption of the venom, and thereby delays the onset of life threatening effects of the venom, thus giving the victim additional time to be transported to a hospital where antivenom can be given.

TEB represents a tumescent solution containing epinephrine and B12 in NS or LR. TEB can function as an excipient for tumescent drug delivery. As an excipient for pre-incisional tumescent drug delivery drugs, (e.g., antibiotics), TEB, which contains B12 or B1 or B6 provides a basis for improving surgical wound healing.

TELB represents a tumescent solution containing epinephrine lidocaine and B12 in NS or LR. TELB is an ideal excipient for pre-incisional tumescent drug delivery. Tumescent lidocaine provides unequalled surgical local anesthesia.

Tumescent Delivery (TD) of a drug (D) is defined as the subcutaneous injection of a relatively large volume of a relatively dilute solution of an injectable water-soluble drug D dissolved in a TEB or TELB solution.

Tumescent Pharmacokinetics & Pharmacodynamics

For dilute concentrations of lidocaine [≤1 gm/L] and epinephrine [≤1 mg/L], there is good, evidenced-based pharmacokinetic data to support the recommended maximum safe mg/kg dosage of TEL lidocaine, which is 28 mg/kg, without liposuction (Klein J A and Jeske D R. “Estimated maximal safe dosage of tumescent lidocaine”, Anesth Analg 2016; 122: 1350-9).

TEL is a 10-fold dilution of commercial 1% lidociane with epinephrine 1:100,000. The recommended maximum safe mg/kg dosage of TEL lidocaine is 28 mg/kg. Because liposuction removes 28% of TEL lidocaine before it can be absorbed into the systemic circulation, the recommended maximum safe dosage of tumescent lidocaine is 45 mg/kg; (Klein J A and Jeske D R. “Estimated maximal safe dosage of tumescent lidocaine”, Anesth Analg 2016; 122: 1350-9). TEL is unique in its ability to achieve wide ambit pure sensory regional anesthesia. The efficacy depends directly upon the bioavailability of TEL within the targeted tissue.

TEL bioavailability is a function of the ambit (extent of exposed tissue) and duration of exposure to lidocaine and epinephrine. Based on clinical experience, the degree of TEL local anesthesia and capillary vasoconstriction is the product of:
1) the concentrations of lidocaine and epinephrine,
2) length of peripheral nerve axons exposed to lidocaine,
3) the volume of capillary-vascularized tissue bathed in TEL, and
4) the duration of TEL exposure.

Detumescence is defined as the process whereby tumescent tissue becomes less swollen and firm. Detumescence is the result of a tumescent solution spreading peripherally along its hydrostatic pressure gradient, thereby flooding a wide ambit of surrounding interstitial tissues with lidocaine, epinephrine and any other drug that has been added to the TEL solution.

Detumescence allows a TEL solution to penetrate both the suprajacent dermis as well as subjacent muscle facia with significant effect. Remarkably, adequate TEL tumescence routinely eliminates the most severe pain of sciatica for 12 to 16 hours.

Detumescence is allows perfect local anesthesia for larges dermatologic surgical procedures. A sufficiently large volume of subcutaneous TEL followed by an adequate time for detumescence, permits pain free excisions of large melanomas with unparalleled surgical hemostasis, and complete postsurgical anesthesia; the capillary vasoconstriction within skin and subcutaneous fat typically eliminates all need for cautery and prolonged local anesthesia-analgesia (≥24 to 48 hours) eliminates 100% of postsurgical opioid requirements.

Adequate Tumescent-Detumescent Anesthesia is an absolute pre-condition for optimal surgical anesthesia, bloodless hemostasis, and complete pain-free incision site post-surgical analgesia. Optimal tumescent surgical anesthesia requires both adequate tumescence and adequate detumescence. An insufficient volume of TEL, insufficient epinephrine and lidocaine concentrations, and insufficient duration of detumescence increases the risk of suboptimal anesthesia, hemostasis and postsurgical analgesia.

B12 in Excipient Solutions for TD

TELB consists of one or more B complex vitamins dissolved in a TEL solution. There is considerable evidence that B complex vitamins, and vitamin B12 in particular, facilitate the healing of surgical wound and peripheral nerve injury.

B12 also improves safety of TEL by giving the dilute solution of lidocaine a distinctive pink color. Such unique color of a solution in an IV bag provides a clear warning to surgical staff by alerting them that the IV bag contains tumescent lidocaine and must never be used for IV infusion. An inadvertent intravenous infusion of the contents of an innocuous unmarked IV bag containing tumescent lidocaine may have caused the unexplained sudden death of at least one liposuction patient who died prior to liposuction during subcutaneous tumescent infiltration after having received only 10 mg/kg of tumescent lidocaine (Rao R B, et al., “Deaths related to liposuction”, N Engl J Med. 1999; 340: 1471-5).

Tumescent Delivery (TD) of a drug (D) is defined as the subcutaneous injection of a relatively large volume of a relatively dilute solution of an injectable water-soluble drug D dissolved in a TEB or TELB solution.

TD is a novel mode of drug delivery which has unsurpassed pharmacokinetic and pharmacodynamic advantages compared to traditional IV, IM and PO modes drug delivery. TEB and TELB are highly effective excipient (carrier) solutions for subcutaneous delivery of drug D.

TD of drug D assures the peak subcutaneous concentration of D that is precisely equal the concentrations of drug D within the TD solution. As a general rule, following subcutaneous tumescent delivery of a drug D, the peak interstitial concentration of D and the bioavailability of D are 10 to 40 times greater and 50 to 100 times greater respectively, compared to IV, IM, or PO delivery of D.

TE represents a tumescent solution containing epinephrine in NS or RL. TE functions as a basic, “barebones” excipient for tumescent drug delivery. But it is not capable of masking the pain-upon-injection caused by some drugs. Nor does it promote wound healing.

TEB represents a tumescent solution containing epinephrine and B12 in NS or LR. TEB can function as an excipient for tumescent drug delivery. As an excipient for pre-incisional tumescent drug delivery drugs, (e.g., antibiotics), TEB, which contains B12 or B1 or B6 improves surgical wound healing. But TEB does not provide local anesthesia.

TELB is a tumescent excipient solution containing epinephrine lidocaine and B12 in NS or LR. TELB is the ideal and uniquely capable excipient for pre-incisional tumescent drug delivery. The lidocaine in TELB is unequalled as a preemptive, intraoperative, and postoperative surgical local anesthetic, a surgical hemostatic, an antiinflammatory, and an antiplatelet anti-thrombotic.

B12 & Niap and Nociception

For surgical procedures, a TELB tumescent solution containing vitamin B12 (≤10 mg/L) provides improves clinical efficacy and safety, compared to TEL. Recent research has established that B12 improves surgical wound healing by encouraging healing following peripheral nerve injury.

Data Supporting the Therapeutic Efficacy of B Vitamins in Wound Healing

There are numerous published peer-reviewed scientific reports, that support the anti-inflammatory and analgesic properties of vitamin B12 with or without B1, B2, or B6.

None of these reports mention the inclusion of epinephrine in the combination of a vitamin B together with epinephrine in the therapeutic composition. Some of these peer-reviewed publications are listed below.

Properties of B12 in Wound Healing

B12 is antiinflammatory. B12 promotes surgical incision wound healing. In skin lesions, the synthesis of free radicals and reactive oxidative species (ROS) by inflammatory cells contributes to the defense against pathogens and mediates important intracellular pathways for the resolution of the inflammatory phase. However, excessive amounts of free radicals and ROS promote tissue oxidative stress, causing deleterious effects on cell membranes, proteins, and nucleic acids (Gonsalves R V, et al. “Cellular and Molecular Mechanisms of Oxidative Stress in Wound Healing”, Oxid Med Cell Longev. 2022; 2022: 9785094).

Vitamin B12 possess antioxidant properties such as: (1) direct scavenging of reactive oxygen species (ROS), particularly superoxide; (2) indirect stimulation of ROS scavenging by preservation of glutathione; (3) modulation of cytokine and growth factor production to offer protection from immune response-induced oxidative stress; (4) reduction of homocysteine-induced oxidative stress; and (5) reduction of oxidative stress caused by advanced glycation end products (Van de Lagemaat E E, de Groot LCPGM, van den Heuvel EGHM. Vitamin B12 in Relation to Oxidative Stress: A Systematic Review. Nutrients. 2019; 11: 482).

B12 for Treating Pain

Vitamin B12 is thought to alleviate surgical pain by promoting myelination, increasing nerve regeneration and decreasing ectopic nerve firing (Julian T, et al. “B12 as a Treatment for Peripheral Neuropathic Pain: A Systematic Review”, Nutrients 2020; 12: 2221). Beneficial effects of vitamin B12 in preventing niap, and preventing and treating pain, include:
Regeneration of nerves
Inhibition of cyclooxygenase enzymes and other pain signaling pathways
Synergistic effects with other pain medications including nonsteroidal antiinflammatory drugs (NSAIDs) and opioids (Buesing S, et al. “Vitamin B12 as a treatment for pain”, Pain Physician 2019; 22: e45-e52.)

Tumescent B vitamins have an anti-nociceptor effect which prevent a potentially noxious event or condition from triggering nociceptor stimulation. In other words, B vitamins can abort the initiation of sensory nerve impulse, and there by prevent the initial generation of a nerve impulse that is perceived as a noxious (painful) event by the central nervous system.

B12 for Treating Zoster

Undiluted B12 injections, consisting of multiple daily intradermal injections of B12 (1 mg/ml) for 2 weeks, have been shown to gradually reduce the pain of acute Herpes zoster (shingles) compared to placebo. The injections, spaced 2 to 3 cm apart, target the most painful areas.

The Herpes zoster virus infects peripheral nerves and causes peripheral nerve injury (PNI).

1) Undiluted B12 plus undiluted plain lidocaine (without epinephrine), when injected using same 2-week protocol of multiple daily injections, results in 30% reduction of zoster pain compared to placebo.

2) Undiluted vitamin B1, when injected using same 2-week protocol of multiple daily injections, results in 30% reduction of zoster-related intensely painful itching compared to placebo.

Our prior US patent [U.S. Pat. No. 10,493,024 B2. Dec. 3, 2019] teaches the use of tumescent solution TEL[AT] containing Epinephrine, Lidocaine, Acyclovir, and Triamcinolone can be used to treat acute Herpes zoster (shingles). Lidocaine in TEL[AT] eliminates the pain of an acute shingles infection for 24 hours or more. Acyclovir is an antiviral drug that stops the replication of the Herpes zoster virus, and limits the PNI caused by the viral infection. Triamcinolone in TEL[AT] decreases the cutaneous inflammation caused by H zoster. Neither lidocaine, acyclovir or triamcinolone contribute directly to the repair of peripheral nerve damaged by H. zoster. B12 (and perhaps B1 or B6) contribute directly to the repair of peripheral nerve damaged by H. zoster.

Anti-Inflammatory Effects of B12

Experimental animal studies have shown that B12 injections into the dermis achieves surprising and unanticipated beneficial therapeutic effects on the cellular components of inflammation. These effects include:
1) decreased neuroinflammation within a surgical wound,
2) increased the rate of repair and healing following physical injury to peripheral nerves axons and Schwann cells following traumatic injury caused by a surgical incision,
3) decreased the effects of proinflammatory macrophages and increased the effects of anti-inflammatory macrophages which thereby facilitate the axonal regeneration following peripheral nerve injuries,

Clinical experience suggests that the anti-inflammatory effects of a solution containing B12, and lidocaine are superior to a solution containing lidocaine alone. Pre-surgical injection of a tumescent epinephrine B12 solution (TEB) into an extensive area of subcutaneous tissue surrounding a surgical incision will promote accelerated rates of wound healing of traumatized tissue, promote repair of surgically transected peripheral nerve and Schwann cells and decrease the risk of prolonged and incomplete tissue repair incidence of severe persistent postsurgical pain.

TELB Yields High Tissue B12 Concentrations

It is known that low B12 serum levels (B12 deficiency) are associated with poor wound healing. Normal physiologic serum concentrations of B12 are less than 0.001 μg/ml.

B12 tissue levels following TELB (cyanocobalamin) infiltration are 10,000 times greater than physiologic serum concentrations (0.001 μg/ml). Prolonged, very high concentrations of tumescent B12 may improve wound healing, or at least reduce the risk of poor wound healing.

NIAP

Nociception refers to neural detection noxious stimuli caused by inflammatory, mechanical, thermal, or chemical events.

Niap (noun) is a neologism defined as any noxious event or condition that activates or causes nociception.

Niapian (adjective) describes an event or condition that is capable of causing niap.

Pain is defined as the conscious awareness of a noxious condition or a niapian event.

Contemporary anesthetics (general anesthetics, nerve block anesthesia, analgesics, opioids, NSAIDs) merely prevent or reduce the awareness of acute pain. Their effects or temporary. When the effects of these drugs wears-off, the perception of pain returns.

There is surgical niap and non-surgical niap. Surgical niap is predictable, preventable, or at least dramatically reduced by TDD. Non-surgical inflammatory niap is treatable.

A physical injury, including trauma to sensory neurons, surgical section, fractures, thermal trauma, mechanical trauma, caustic chemical trauma) is a form of niap.

An inflammatory injury (primary cytokine-mediated tissue inflammation, including infection-associated inflammation).

Secondary inflammation following tissue trauma is a form of niap.

Niap is preventable. Surgical incisions cause niap. Surgical niap is readily prevented by pre-incisional subcutaneous infiltration of tumescent-detumescent epinephrine, lidocaine, B12, in combination with antibiotics Eliminating acute incision-site surgical pain, and incision site infection dramatically reduces the risk of persistent postsurgical pain (PPP).

Preventing surgical niap requires: 1) safe combination of and effective drugs, and 2) a safe and effective mode of drug delivery.

Surgical incisions involve both primary physical traumatic injury and secondary inflammatory injury. Both modes of injury are noxious events or conditions which trigger nociceptors and initiate afferent impulse conduction along peripheral sensory nerves toward the central nervous system (CNS).

Tumescent Niap Block prevents the occurrence of niap, niapful inflammation or niapian trauma.

Tumescent Delivery (TD) of drugs is the first only, mode of drug delivery that can preemptively prevent niap and terminate on going niap. TD is an example of contra-niap.

At present, the standard modes of treating pain involve the use of pain-blocker drugs such as general anesthesia (GA), nerve block anesthesia (NBA), anesthetic/analgesics (ketamine), sedatives (propofol), opioids, and commercially available local anesthetics, nonsteroidal anti-inflammatory drugs (NSAIDs). Currently available modes of drug delivery that treat pain and (GA, NBA, IV, IM, PO) merely block the perception of pain. Pain-blocker drugs are not intended nor capable of blocking niap.

There are no off-the-shelf commercial versions of drug formulations that can function as niap-blocker drugs. All anti-niap drug formulations have been invented by Jeff Klein specifically for the prevention and treatment of niap. Incision-site niap typically involves extensive volumes peripheral subcutaneous tissues. Thus, any effective anti-niap drug treatment must be able to deliver the drugs into a wide ambit of subcutaneous tissues. Tumescent delivery (TD) was invented by Jeffrey Klein, specifically to provide this ability. Tumescent delivery (TD) is the only safe, effective mode of subcutaneous interstitial delivery of niap-blocker drugs.

Tumescent effects: large volume reservoir effect, maximal interstitial penetration along hydrostatic pressure gradients, rapid wide ambit (volume) spread through throughout the targeted cutaneous tissue and adjacent musculoskeletal tissues.

The TD Pump™ is a digitally controlled, peristaltic roller pump that provides high precision fluid flow rates in increments of 1 ml/min at 1 RPM up to more than 700 ml/min at 600 RPM.

The Klein TD pump™ allows this slow and precise flow rate control allows the initial use of the tiniest hypodermic needles (23 gauge×4 mm) to achieve virtually painless intradermal and subcutaneous tumescent drug delivery. High precision fluid flow rate control permits pure sensory random area regional anesthesia for any cutaneous, subcutaneous, and adjacent musculoskeletal tissues anywhere on the body.

HK Klein tumescent delivery TD pump™ and HK Infiltration tubing were specifically designed for treating Herpes zoster.

Necrotizing Inflammation

Tumescent drug delivery eliminates pain and prevents progression of necrotizing inflammation in pathologic conditions involving capillary destruction or occlusion where traditional systemic drug delivery is insufficient. Examples: Ecthyma gangrenosum, pyoderma gangrenosum, neutrophilic dermatoses, calciphylaxis

Bone Fracture Pain and Localized pain syndromes (sciatica) are easily and effectively treated with TELB.

Tumescent Effects

Detumescent effects: Time-dependent spread of tumescent solution.

Epinephrine effects: profound capillary vasoconstriction, delayed systemic absorption, augmented bioavailability of drug, and prolonged local effect of all drugs in the TELB solution including epinephrine, lidocaine and B12.

Lidocaine effects: safest of all local anesthetics, eliminates pain upon injection of any injectable drug, blocks nociceptor function, anti-inflammatory, bactericidal, inhibits antiplatelet activation, etc.

Tumescent lidocaine is both more effective and far safer than tumescent bupivacaine. Tumescent compositions and methods of using tumescent compositions, including specific compositions, are documented by numerous patents, including U.S. Pat. Nos. 8,957,060, 9,623,030, 10,322,083, 11,213,481, 10,493,024, 11,696,890, 11,723,859, 11,241,412, and 10,786,483.

TD permits the painless, safe, effective, efficient unprecedented subcutaneous interstitial infiltration of large volumes, (1 to 4 liters or more) therapeutic tumescent solutions. Tumescent delivery of drugs is a unique mode of drug delivery. TD achieves pharmacokinetic and pharmacodynamic effects for targeted localized subcutaneous drug delivery that are far superior to the capabilities of IV, IM, or PO modes of drug delivery.

The local subcutaneous delivery of a tumescent solution containing therapeutic drugs achieves (a) a localized effect or (b) generalized effect on tissue distant from the site of the injection by means of an abscopal effect on immunologic processes that cause acute or chronic inflammation or neuropathic pain.

The abscopal effect is a systemic immune response mediated by the effects of a localized or targeted therapy, such as radiation therapy or injection of an antiinflammatory immunomodulator.

As a novel mode of drug delivery, TD required the development of novel devices, also invented by Dr. Jeff Klein, that have allowed the safe, effective, efficient and painless subcutaneous infiltration of tumescent solutions.

HK Klein TD Pump™ & Technique of TELB Injection

The new HK Klein TD Pump™ was invented and specifically developed for painless, safe, effective, and efficient Tumescent Delivery of niap-blocker drugs (NBDs).

The Klein TD Pump™ provides painless, safe, and effective tumescent drug delivery into cutaneous tissues.

Using the Klein TD Pump™, 100% of the most severe acute Herpes zoster pain affecting any dermatome, can be eliminated within minutes. If the tumescent solution contains triamcinolone and acyclovir, TELB [AT], acute cases of H. zoster are cured with only one to three daily infiltrations.

The Klein TD pump™ allows painless intradermal and subcutaneous tumescent infiltration of all affected area of any trigeminal dermatome. Herpes zoster ophthalmicus affecting the scalp, eyelids, and nose is safely and painlessly infiltrated using 32 g×mm, 30 g×12 mm, 25 g×15 mm and 22 g×25 mm in a sequential fashion. TELB[AT] solution is infiltrated above eyebrow and outside the orbital rim, the tumescent hydrostatic pressure gradient gently pushes the TEL[AT] solution through the palpebral interstitium. Herpes zoster oticus is caused by the spread of the Herpes varicella-zoster virus to facial nerves. It is characterized by intense ear pain, a rash around the ear, mouth, face, neck, and scalp and paralysis of facial nerves. Other symptoms may include: hearing loss, vertigo (abnormal sensation of movement), tinnitus (abnormal sounds), loss of taste, dry mouth and eyes.

The HK Surgical Klein Tumescent Delivery Pump was specifically designed to provide precision intradermal tumescent drug delivery of antimicrobials, such as TELB [antimicrobials].

When a drug D is dissolved in a TE solution, the resulting solution is represented by TE[D], where D can be any injectable drug, including epinephrine itself.

A subcutaneous injection of a relatively large volume of a TE[D] solution produces widespread capillary vasoconstriction within the infiltrated tissue which delays the systemic absorption of any drug D. This delayed absorption of D prolongs the local effect of D in the targeted subcutaneous tissue. The subcutaneous tumescent delivery (injection) of D significantly increased the local subcutaneous bioavailability of D.

Following the subcutaneous injection of the TE[D] solution, the concentration of drug D in the subcutaneous interstitial tissue is precisely the concentration of drug D in the TE[D] solution.

The concentration of drug D in the targeted subcutaneous interstitial tissue is far greater than the concentration of drug D than can be achieved by any other mode of drug delivery, including intravenous (IV), intramuscular (IM), or oral (PO) drug delivery.

In addition, the effective implementation of TD of injectable drugs requires the unique and newly developed digitally controlled, high precision tumescent infiltration peristaltic roller pump, the HK Klein tumescent delivery TD-Pump™. The HK Klein TD-Pump™ is sold by HK Surgical, Inc., San Clemente, CA 92673, www.hksurgical.com., which was founded 25 years ago by Jeffrey Klein. Jeff Klein invented the tumescent technique for local anesthesia in 1985, published the first description of the tumescent technique in 1987 (Klein J A. The tumescent technique for liposuction surgery. J Am Acad Cosmetic Surg, 1987; 4:263-267).

Klein invented the first tumescent infiltration tubing in 1990. The HK Klein TD-Pump™ is the 4^{th} generation tumescent infiltration pump to have been designed by Jeffry Klein.

The HK Klein TD-Pump™, is unique in its ability to allow a clinician to precisely select the pump's fluid flow rates in increments of 1 RPM, over a range of 1 RPM to 600 RPM. Virtually every other tumescent infiltration pump has been designed and solely intended for large volume subcutaneous infiltration at pump rates greater than 40 RPM specifically for rapid subcutaneous infiltration of tumescent epinephrine lidocaine (TEL) using a large bore infiltration cannula for tumescent liposuction under general anesthesia. Such pumps do not allow for the gentleness and precision required for using the smallest hypodermic needles. The HK Klein TD-Pump™ allows the use of the tiniest hypodermic needles (32 gauge×4 mm and 30 gauge×4 mm) in order to painlessly initiate tumescent infiltration and then gradually advance to large hypodermic needles (25 gauge, 22 gauge and 20 gauge) to provide rapid tumescent drug delivery into any accessible cutaneous or mucosal surface.

B12 and B2 Function as Color Indicators

The following three scenarios illustrate common situations where color-indicators in tumescent solutions such as TEL have significant utility.

1. Surgical Operating Rooms

TEL is an off-label use of lidocaine because its mg/ml drug concentrations are off-label, and the recommended maximal safe mg/kg drug dosages are off-label. As the consequence of TEL being off-label: (1) there are no CPT codes for providing tumescent anesthesia, (2) insurance companies do not reimburse physicians for the time-consuming effort of injecting TEL, and (3) it is the surgeon who injects tumescent anesthesia as part of the surgical procedure. Surgeons are becoming more aware that TEL provides outstanding local anesthesia and exceptional surgical hemostasis. However, surgeons do not receive insurance payments for the time and effort required to provide TEL. Because they are not paid for providing TEL, anesthesiologists generally have little interest in TEL or knowledge about the benefits of TEL which include superior pre-emptive analgesia, postoperative acute pain control, reduced postoperative opioid requirements, reduced risk of persistent postoperative pain (PPP) syndromes, and reduced risk of surgical site infections.

It is reasonable to assume that every operating room (OR) staff person in any OR is a “person skilled in the art” of intravenous infusion. Yet virtually none of them would ever suspect that an unmarked IV bag of saline might contain a drug that is not safe for intravenous administration.

Consequently, few operating room personnel are aware of the potential risks for an inadvertent intravenous infusion of TEL. It is unexpected, and nonobvious to “a person skilled in the art” of IV drug delivery, that an unmarked IV bag of normal saline found in an operating room might be lethal if injected IV. An accidental IV infusion of TEL is a prevalent but unrecognized safety threat. A reliable solution is nonobvious.

2. Mass Casualty and Combat Injuries

Mass casualty injuries and mass combat wound injuries can overwhelm first responders. Multiple victims may simultaneously require wound hemostasis, significant intravascular fluid replacement, and burn resuscitation. Fluid replacement often requires the intravenous delivery of normal saline or lactated Ringer's solution. Establishing intravenous access for IV fluid replacement is not always feasible. Enough personnel who are skilled at inserting an intravenous catheter may not be present. Establishing intravenous access at night, in conditions of poor visibility, or in midst of combat is clearly problematic.

Subcutaneous infiltration of large volumes of dilute TEL, for example containing an antibiotic: 1) is relatively easy to achieve by nearly any layperson simply by inserting a subcutaneous catheter and injecting the TEL fluid, 2) provides excellent local anesthesia for pain relief to blast and burn victims, 3) provides profound local tissue hemostasis for open wounds including non-compressible wounds on the torso, and 4) delivers antibiotics to wounds and prevents subsequent infection and sepsis. In the chaos of a mass casualty, the concomitant presence and utilization of IV bags of saline and IV bags of TEL presents a real risk of inadvertent “wrong IV bag” administration. We must have a reliable means of rapidly identifying an IV bag of TEL.

3. Prehospital Treatment of Snakebite Envenomation

Every year, venomous snakebites account for 140,000 deaths worldwide with 400,000 amputations or permanent disabilities (Bhaumik S, Beni D, Lassi Z S, Jagnoor J. Interventions for the management of snakebite envenoming: An overview of systematic reviews. PLoS Negl Trop Dis. 2020; 14(10): e0008727). There is no effective pre-hospital treatment for snakebites. We have shown that the subcutaneous injection of large volumes of dilute TEL at the anatomic site of a snakebite causes profound local vasoconstriction, resulting in delayed systemic absorption of the venom and prolonged survival, giving victims additional time to be transported to a hospital and receive antivenom therapy (Makdisi J R, Kim D P, Klein P A, Klein J A. Tumescent contravenom: murine model for prehospital treatment of Naja naja neurotoxic snake envenomation. Int J Dermatol. 2018; 57:605-610). A TEL solution in an IV bag which contains drugs that either delay systemic absorption of venom or neutralize venom in situ is known as tumescent contravenom. Tumescent solution that contains snake venom-neutralizing protease inhibitor drugs, can neutralize the venom of a broad spectrum of snake species across all geographic regions.

Snakebite envenomations often occur in rural, medically indigent populations, where there are numerous unrelated dialects, and literacy rates are low. IV bag of tumescent contravenom with a label warning against its IV infusion, is of little benefit if the first responder is illiterate or cannot read the printed language on the label. There is an obvious need for a means of rapidly distinguishing between an IV bag of tumescent contravenom and IV bag of saline.

The Risks of Human Error with TEL

Virtually all TEL-associated complications are the result of human error occurring 1) during TEL preparation or 2) during tumescent drug delivery.

Preparation Errors: Worldwide, every time a TEL solution is required, it must be prepared extemporaneously by an individual person who may not be well trained or experienced. There are no well-defined set of qualifications for persons who prepare TEL solutions. Complications resulting from preparation of TEL solutions include errors in formulation of TEL solutions such as 1) wrong-amounts lidocaine or epinephrine, and 2) contamination of the TEL solution due to poor aseptic technique. These types of complications suggests that TEL ought to be commercially available as a USP preparations manufactured with the highest Good Manufacturing Practices (GMP) standards.

At present, TEL is not commercially available as an FDA-approved, USP solution. Any commercial version of TEL will require an approved FDA investigational new drug (IND) application and ultimately an approved FDA new drug application (NDA). A liter of TEL contains 1 gm of lidocaine which is potentially fatal if given by rapid IV infusion. There are no standardized safety measures that minimize the risk of inadvertent IV delivery of TEL and the consequent risk of a fatal lidocaine overdose.

Delivery Errors: The method of delivery of TEL can cause physical injury to patients. For example, sharp-tipped, hypodermic needle-like infiltration cannulas can lacerate vessels and nerves. Exceptionally long sharp-tipped infiltration cannulas in the hands of naïve or careless personnel have resulted in significant injuries such as pneumothorax and injury to abdominal viscera. The use of blunt-tipped infiltration cannulas mitigates against traumatic laceration of tissue targeted for tumescent infiltration.

When tumescent infiltration is accomplished under general anesthesia, there is an increased risk of injury to the patient. General anesthesia has a permissive effect on rapid tumescent infiltration, often with little gentleness or attention to detail. Under general anesthesia, a patient cannot alert the surgeon if the infiltration cannula has been inserted too deeply into muscle, the abdominal cavity, or a major vessel. It is safer to provide tumescent local anesthesia in a fully awake patient. With sufficient training and finesse, tumescent anesthesia can be achieved painlessly and more safely when done totally by local anesthesia.

Medical devices have been designed by the inventor which improve the process and safety of tumescent infiltration. The “Klein Pump®” is a tumescent peristaltic roller pump and tubing that permits fluid to be transferred at a precisely controlled rate from an IV bag of a TEL solution directly into targeted subcutaneous tissue. The Klein Pump® is the industry standard for efficient, accurate tumescent infiltration. The tumescent peristaltic roller pump and tubing were originally designed and popularized by Klein in 1989. Prior to the Klein Pump®, Klein had invented a syringe-assisted a fluid-transfer device with one-way check-valve. This Klein Handle permits fluid to be efficiently transferred from an IV bag into a syringe. After the fluid has been injected into subcutaneous tissue, the syringe is refilled by merely retracting the syringe plunger (Klein J A. The tumescent technique for liposuction surgery. J Am Acad Cosmetic Surg, 1987; 4:263-267). The tumescent peristaltic pump and the tumescent fluid transfer syringe with check-valve improved efficiency and improved safety by eliminating the task of repeatedly refilling multiple syringes. U.S. Pat. No. 5,052,999 describes liposuction methods and apparatuses.

The inventors are pursuing an FDA investigational new drug (IND) application for tumescent epinephrine lidocaine with vitamin B12 and/or vitamin B2 (TELB). It is likely that TELB will eventually become widely recognized as an ideal excipient (carrying solution) for tumescent drug delivery (TDD) for a wide variety of therapeutic drugs. The pharmacokinetic profile of TDD has many important advantages for specific cutaneous and muscular therapeutic targets compared to the traditional IV, IM, and PO modes of drug delivery. For many years, TEL was merely conceived of as a safe and efficient technique for providing local anesthesia to an exceptionally large area of skin and subcutaneous tissue. A few years ago, Klein discovered that TEL functions as an excellent excipient for subcutaneous tumescent drug delivery of numerous therapeutic drugs. For many disease conditions, TDD is a more effective and safer mode of drug delivery than IV, IM or PO drug delivery.

U.S. Pat. No. 10,493,024 B2 describes the use to subcutaneous tumescent delivery of acyclovir and triamcinolone to rapidly eliminate the most severe pain of acute Herpes zoster (Shingles) infection, using a solution of Tumescent Epinephrine Lidocaine Triamcinolone Acyclovir (TELTA). The subsequent addition of vitamin B12 resulted in TELTAB, which further improved this unprecedentedly effective treatment. In U.S. Pat. No. 10,493,024 B2 Klein defined tumescent drug delivery as follows:

Tumescent drug delivery (TDD) consists of the subcutaneous injection of a relatively large volume of a relatively dilute solution of a drug D dissolved in a dilute tumescent solution of epinephrine (1 mg/L) in 0.9% physiologic saline. When the primary therapeutic target for a drug is cutaneous, subcutaneous, or musculoskeletal tissue, the pharmacokinetic profile of TDD is often far superior to that of intravenous (IV), intramuscular (IM) or oral (PO) drug delivery. To be considered a good candidate for tumescent drug delivery, the concentration of a drug D in a tumescent solution must be:
1) below the threshold for local subcutaneous tissue toxicity
2) above the threshold for positive local therapeutic effect
3) above a concentration safely achievable by intravenous (IV), intramuscular (IM) or oral delivery.
4) compatible with other drugs in the TDD solution.

Examples of TDD are described in several recent U.S. patents and publications: U.S. Pat. No. 9,623,030 (Tumescent Antibiotic Solution); U.S. Pat. No. 10,322,083 B2 (Tumescent Drug Delivery), U.S. Pat. No. 10,786,483 B2 (Tumescent Contravenom Drug Delivery), U.S. Application Publication No. 2019/0240 A1 (Tumescent Infiltration Drug Delivery of Cannabinoids) and U.S. Application Publication No. 2019/0240189).

Klein has submitted additional TDD-related patent applications. These involve the use of TDD of biologic drugs for treating cancers and inflammatory diseases. It is likely that TDD will soon gain widespread acceptance and application. In the future, as the use of TEL and TDD become more commonplace, the need for TEL related safety measures will become urgent.

Warning-Labels are Insufficient: Warning-labels on the packaging of commercial drugs are commonplace. It is obvious that a warning-label on a bag of TEL will lessen the risk of an inadvertent IV infusion of TEL. A prominent warning-label affixed to every IV bag containing TEL has been advocated by Klein for many years. However, awareness among clinicians of the safety concerns surrounding the clinical use of TEL remains limited. Few surgical facilities have written policies and procedures that require the use of a warning label on bags of TEL. Any expectation that surgical facilities will ever voluntarily promote the use of TEL warning-labels is overly optimistic and unrealistic.

Implementation industry wide of TEL warning-labels and TEL color-indicators on IV bags containing TEL cannot be voluntary. Eventually they must be features of commercially available IV bags of TEL. The uniform use of warning-labels and color-indicators in IV fluids will only be achieved by the universal availability of commercially manufactured TEL solutions. IV bags containing TEL must become available as GMP manufactured and FDA-approved products.

Unexpected Benefit of a Color-Indicator for an IV Solution

There is no reason why an IV bag of saline, containing a drug intended for IV delivery, would ever need to have a color-indicator dye added to the solution.

There is not a single example of a color-indicator additive to be used together with any FDA approved drug intended for IV infusion. In fact, the FDA has never approved a colored dye which has “color-indicator for an IV solution” as its indication.

It is common sense that a drug in an IV bag, prepared by a hospital pharmacy, would naturally be intended for IV delivery.

It is nonobvious that anyone “skilled in the art” of using IV bags or “skilled in the art” of administering drugs by IV infusion would ever contemplate the addition of a color-indicator dye to any IV bag containing a saline solution.

We have identified two FDA-approved, essentially non-toxic, water-soluble therapeutic vitamins which impart a colored appearance, B12 (pink) and B2 (yellow), when dissolved in an aqueous solution. B12 and B2 promote regeneration of injured nerve fibers and wound healing (Mota I G, Das Neves RAM, Nascimento S S, Maciel B L L, Morals A H, Thais Souza Passos T S. 2021; Artificial Dyes: Health Risks and the Need for Revision of International Regulations, Food Reviews International). Neither B12 nor B2 have ever been used as color-indicators to warn against IV infusion of a solution contained in an IV bag.

B12 and B2 are both considered off-label when the indication is 1) color-indicator for therapeutic solutions intended only for subcutaneous infiltration or topical application and 2) to warn against an inadvertent intravenous infusion. We are in the process of submitting an FDA investigational new drug (IND) application for the use of B12 and/or B2 as a color-indicator in tumescent epinephrine lidocaine solutions (TEL). TEL is only safe for subcutaneous infiltration or topical applications (see FDA IND #155261).

The pharmacologic aspects of TEL are extremely safe. To the best of my knowledge, there are no reports of TEL toxicity when TEL is “used as directed” in numerous peer-reviewed publications authored by Klein. Whenever there has been a TEL-associated complication, it has been the direct result of human error. For example: 1) Ignoring accepted recommendations for maximal safe mg/kg lidocaine dosage for tumescent lidocaine, 2) human errors in the extemporaneous formulation of TEL solution (e.g. using the correct volume of “a commercial solution of lidocaine with epinephrine for infiltrative local anesthesia” but mistakenly using 2% lidocaine with epinephrine instead of 1% lidocaine with epinephrine, thereby exceeding (doubling) the intended mg/kg dosage, 3) injuries due to the use a long sharp-tipped infiltration cannula instead of safer, blunt-tipped infiltration cannula, 4) administering the TEL solution by an inadvertent IV infusion instead of subcutaneous infiltration.

For the past 35 years, Klein has been continuously concerned about the risk of an inadvertent IV delivery of a TEL solution. He has often published and have repeatedly advocated the use of warning-labels that state, “NOT for IV—For Subcutaneous Injection Only” (Klein J A, Langman U. Prevention of Surgical Site Infections and Biofilms: Pharmacokinetics of Subcutaneous Cefazolin and Metronidazole in a Tumescent Lidocaine Solution. Plast Reconstr Surg Glob Open 2017; 5, e1351). However, warning labels are insufficient and far from foolproof. Among the potential problems with warning labels are: 1) The application of warning-labels on every bag of TEL might simply be ignored or overlooked by the individual who is extemporaneously mixing the solution, 2) Not everyone in a surgical operating room can be expected to be fluent in the language of the written warning-label, 3) Warning-labels can become detached from the IV bag, 4) A supply of printed safety labels may simply not be available.

There is a critical need for a reliable method that allows rapid, error-free identification of an IV bag of saline that contains TEL. Our novel remedy to this urgent problem involves a nonobvious addition of vitamin B12 (cyanocobalamin) or vitamin B2 (riboflavin) to the TEL solution. Cyanocobalamin is a man-made form of vitamin B12 that is commercially available for intramuscular (IM) injection. Cyanocobalamin for injection is always given by IM injection because IV delivery of cyanocobalamin results in the vitamin being rapidly excreted in the urine.

It is nonobvious that a color-indicator dye for TEL would ever be a thought of as a realistic consideration. During the entire 35 years since the introduction of TEL utilization, no one has ever suggested using a color-indicator for TEL. Hundreds of thousands of surgeons use TEL daily, yet no one has proposed the use of a color-indicator for TEL. It is even more nonobvious and unexpected that a TEL solution could include a color-indicator dye that also has therapeutic properties relevant to a local anesthetic. Even if someone had contemplated the utility of a color-indicator dye for use in an IV bag, there is no FDA-approved chemical that has “color-indicator for IV solutions” as an approved indication. The use of vitamin B12 (cyanocobalamin) or vitamin B2 (riboflavin) as a therapeutic additive and dependable color-indicator to TEL solutions is unexpected and nonobvious.

Vitamins B12 and B2 are nontoxic color-indicators, which when added to a drug solution in an IV bag, warn against IV infusion of that drug. The B vitamins consist of 8 water soluble vitamins: thiamin (B1), riboflavin (B2), niacin (B3), pyridoxine (B6), cobalamin (B12), folic acid, pantothenic acid, and biotin. B vitamin deficiencies adversely affect wound healing. B vitamins are required for collagen formation, optimal immunity, and protein, DNA and red blood cell synthesis. There is evidence that B12 and B2 improve wound healing.

Because of their rapid real excretion, vitamins B12 and B2 do not accumulate in tissues at toxic concentrations. Both B12 and B2 are safe color-indicators for IV bag solutions.

B12 and B2 have significant therapeutic effect. Vitamin B12 and vitamin B2 are both necessary for wound healing (Ehmedah A, Nedeljkovic P, Dacic S, Repac J, Pavlovic B D, Vucevic D, Pekovic S, Nedeljkovic B B. Vitamin B Complex Treatment Attenuates Local Inflammation after Peripheral Nerve Injury Molecules. 2019; 24: 4615; Baltrusch S. The Role of Neurotropic B Vitamins in Nerve Regeneration. Biomed Res Int. 2021; 2021: 9968228; Julian T, Syeed R, Glascow N, Angelopoulou E, Zis P. B12 as a Treatment for Peripheral Neuropathic Pain: A Systematic Revie. Nutrients. 2020; 12: 2221; and Suwannasom N, Kao I, PruB A, Georgieva R, Balmier H. Riboflavin: The Health Benefits of a Forgotten Natural Vitamin. Int J Mol Sci. 2020; 21: 950).

B12 and B2 can each act as dependable visual warning-indicators against inadvertent IV delivery of TEL. When 10 mg of B12 (equivalent to 10 ml of USP vitamin B12 for injection) is added to a 1,000 ml bag of saline, it imparts a distinctive pink color to the solution. B2 imparts a distinctive yellow color to a saline solution. USP B12 and USP B2 for injection are each FDA approved for systemic delivery by injection. B12 and B2 have no significant drug interactions. For short term therapy and at the suggested dosages, neither B12 nor B2 are associated with significant toxicities.

By convention, TELB can designate a TEL solution containing either B12 or B2 or both B12 and B2.

When greater specificity is required, combination of TEL+vitamin B12 is designated by TELB₁₂ and the combination of TEL+vitamin B2 is designated by TELB₂.

Vitamin B12 may be used in tumescent solutions at concentrations of 1 mg/L to 500 mg/L, including concentrations of 1 mg/L, 5 mg/L, 10 mg/L, 15 mg/L, 20 mg/L, 25 mg/L, 30 mg/L, 35 mg/L, 40 mg/L, 45 mg/L, 50 mg/L, 55 mg/L, 60 mg/L, 65 mg/L, 70 mg/L, 75 mg/L, 80 mg/L, 85 mg/L, 90 mg/L, 95 mg/L, 100 mg/L, 150 mg/L, 200 mg/L, 250 mg/L, 300 mg/L, 350 mg/L, 400 mg/L, 450 mg/L and 500 mg/L.

Vitamin B2 may be used in tumescent solutions at concentrations of 10 mg/L-5 g/L, including concentrations of 10 mg/L, 15 mg/L, 20 mg/L, 25 mg/L, 30 mg/L, 35 mg/L, 40 mg/L, 45 mg/L, 50 mg/L, 55 mg/L, 60 mg/L, 65 mg/L, 70 mg/L, 75 mg/L, 80 mg/L, 85 mg/L, 90 mg/L, 95 mg/L and 100 mg/L, 200 mg/L, 300 mg/L, 400 mg/L, 500 mg/L, 600 mg/L, 700 mg/L, 800 mg/L, 900 mg/L, 1 g/L, 1.5 g/L, 2 g/L, 2.5 g/L, 3 g/L, 3.5 g/L, 4 g/L, 4.5 g/L and 5 g/L.

Although Klein invented TEL, and he is an NIH-trained clinical pharmacologist, the idea of adding a color-indicator to TEL solutions occurred to him only recently. Klein is one of the most knowledgeable experts concerning the pharmacology of subcutaneous tumescent drug delivery. After completing an NIH research fellowship in clinical pharmacology, he invented TEL in 1985 (Klein J A. The tumescent technique for liposuction surgery. J Am Acad Cosmetic Surg, 1987; 4:263-267). He has written a 500-page book about TEL published in 2000 (Klein J A, Tumescent Technique: Tumescent Anesthesia & Microcannular Liposuction. Mosby, St. Louis, 500 pages. March, 2000). He has published numerous peer-reviewed journal articles about TEL. He has lectured nationally and internationally about the maximal safe mg/kg dosages of TEL. He has obtained multiple US patents involving TEL. Yet it in 35 years of research and development of TEL, it had never occurred to him to add a color-indicator to every bag of TEL for the purpose of providing a warning against an inadvertent intravenous administration of TEL.

In all years his of experience as a physician, surgeon, and pharmacologist, the saline solution in every IV bag in every operating room has always been as clear as water. There has never been an IV bag containing colored saline. Further, there is no obvious reason why anyone would ever want to put a colored dye into an IV bag of saline. In fact, there is no FDA approved color indicator for solutions intended for IV delivery. It had never occurred to him that there could be any utility in identifying a safe color-indicator for inclusion in IV bags containing saline.

Tumescent Infiltration Drug Delivery

Disclosed herein are methods that utilize as tumescent infiltration (TI) drug delivery. TI comprises a novel mode of drug delivery having a unique multi-compartment pharmacokinetic performance and presenting unique therapeutic opportunities. From a pharmacokinetic perspective, TI is functionally distinct from IV (intravenous), IM (intramuscular), PO (per os, oral), topical (percutaneous) and simple subcutaneous injection.

After tumescent infiltration, subcutaneous interstitial fluid (ISF) is designated tumescent interstitial fluid (TISF). Infiltration refers to drug delivery by injection of a liquid into a tissue. Infusion refers to drug delivery by pouring a liquid into a vein.

TI drug delivery is the direct subcutaneous infiltration of drug(s) dissolved in a large volume of a physiologic crystalloid solution such as 0.9% physiologic saline or lactated Ringer's solution. Depending on the clinical objective, a TI solution may contain a dilute vasoconstrictor (e.g., epinephrine) for delayed systemic absorption or a dilute capillary vasodilator (e.g., lidocaine or niacin) for rapid systemic absorption. (see, U.S. Pat. No. 7,572,613).

TI drug delivery provides unique subcutaneous and systemic concentration-time profile (bioavailability) of a wide range of drugs (antibiotic, antiviral, antifungal, anticancer, analgesic, local anesthetic, biologic, etc.) following subcutaneous tumescent infiltration (TI) drug delivery. Specifically, TI simultaneously produces:
1) Subcutaneous concentrations of a drug that are safe but also far exceed concentrations achievable by any other mode of drug delivery.
2) Systemic (serum) concentrations of the drug with a concentration-time profile resembling a slow constant IV infusion.

TI comprises a combination of a unique delivery vehicle with unique properties and a drug delivery method. TI drug delivery consists of a drug (D) dissolved in a dilute tumescent solution that typically consist of lidocaine (≤1 gm/L), epinephrine (≤1 mg/L), sodium bicarbonate 10 mEq/L in 0.9% physiologic saline. Alternative embodiments of TLA can involve higher or much lower concentrations of these components and/or alternative local anesthetics.

TI drug delivery permits safe and effective local (subcutaneous or deep tissue) infiltration of a large dose of a drug in a large volume of a dilute solution, which otherwise could not be injected because of dose-related systemic toxicity (typically manifested as pain, inflammation or necrosis.

One embodiment of TI drug delivery consists of a large volume of dilute tumescent drugs injected subcutaneously. In another embodiment, a small volume of dilute tumescent anti-tumor drugs is injected directly over a certain time interval, often prolonged, into a deep parenchymal tissue to target a malignant neoplasm.

Examples of specific embodiments of TI drug delivery include:
1) prevention of surgical site infections,
2) treating necrotizing soft tissue infections where IV antibiotic delivery may be inadequate because of blood vessel necrosis,
3) treating localized cutaneous infections in immunocompromised patients,
4) tumescent infiltration acyclovir delivery for treating Herpes zoster,
5) tumescent antifungal delivery for local treatment of cutaneous superficial or deep fungal infections with a relatively high local drug concentrations while significantly reducing the peak serum concentration associated with nephrotoxic, hepatotoxic and ototoxic drugs,
6) tumescent anti-neoplastic delivery for treating cutaneous and subcutaneous malignancies or metastases, for example “in-vivo gene transfer” or biologic drug delivery targeting pancreatic adenocarcinoma,
7) tumescent delivery of a biologic drug consisting of a large molecule snake antivenin for targeting the toxic venom proteins as they are absorbed via lymphatic vessels.

Specific Advantageous Features of Tumescent Drug Delivery

Tumescent Infiltration Drug Delivery has a unique ability to achieve both a relatively high prolonged local drug concentration within the tumescent subcutaneous tissues as well as a prolonged slow constant systemic absorption of drugs from the tumescent tissues into the systemic circulation, where the pharmacokinetic profile of the systemic absorption resembles a slow constant IV infusion. This unique feature of tumescent infiltration (TI) cannot be matched by any other mode of drug delivery. TI is a novel mode of concomitant prolonged local and prolonged systemic drug delivery with unanticipated therapeutic benefits.

Incremental dilution of an arbitrary drug D progressively reduces its subcutaneous toxicity. For any given commercial formulation of a water-soluble drug D that is toxic when injected into subcutaneous tissue, there is always a sufficient tumescent dilution of drug D that is not toxic upon subcutaneous injection.

The definition of TTAR: The tumescent therapeutic ambit range (TTAR) of a drug D is defined as the range of the drug's concentrations within a tumescent solution, such that the tumescent concentration of D is simultaneously:
1) below the threshold for local tissue toxicity,
2) above the threshold for positive local therapeutic effect,
3) above the concentration achievable by IV, IM or PO delivery.

It is to be understood that a concentration threshold for a drug effect is rather nebulous. A precise definition would require a statistical estimate of a safe dose using statistical tolerance interval analysis. For example, “safe” is defined as the estimated dose where, with 95% confidence, the risk of a mild toxic event after a single dose is than 1/1,000.

In some embodiments, the subcutaneous concentration of the drug, or therapeutic agent, achieved is from about 1-100 times the maximum subcutaneous interstitial fluid concentration that can be achieved by conventional intravenous delivery or oral delivery of the drug or therapeutic agent. In some embodiments, the subcutaneous concentration of the drug or therapeutic agent achieved is from about 2, 3, 4, 5, 6, 7, 8, 9, 10, 15, 20, 25, 30, 35, 40, 45, 50, 55, 60, 65, 70, 75, 80, 85, 90 or 95 times the maximum subcutaneous interstitial fluid concentration that can be achieved by conventional intravenous delivery or oral delivery of the drug or therapeutic agent.

Some drugs may not have a tumescent therapeutic ambit range. For example, a sufficiently safe dilution may be too dilute to have a positive therapeutic effect; or the subcutaneous bioavailability of the drug by TI is equal to that of either IV, IM or PO delivery.

Some drugs are inherently painful upon injection. Dilute Tumescent infiltration of a drug is less painful because dilute drug is less painful than more concentrated drug solutions and because dilute lidocaine in the solution eliminates the pain caused by subcutaneous delivery.

In certain clinical situations tumescent lidocaine (high prolonged wide-spread local subcutaneous concentrations) provides important unanticipated therapeutic benefits that are not available with IV, IM or oral delivery. For example, tumescent lidocaine (at concentrations that are significantly higher than can be safely achieved by IV, IM or PO delivery) has antibacterial, antithrombotic and anti-inflammatory properties. These are a unanticipated unique features of TI drug delivery that IV, IM, and PO delivery do not provide.

A lidocaine component of TI drug delivery can provide pain relief for pain associated with a disease being treated by TI. For example, TLA can relieve the acute pain associated with Herpes zoster. This is a unique feature of TI drug delivery that IV, IM, and PO delivery do not provide.

Dilute epinephrine in the TI solution induces intense prolonged local subcutaneous capillary vasoconstriction. This TI vasoconstriction accounts for uniquely beneficial pharmacokinetic properties. The TI local vasoconstriction profoundly delays the systemic absorption of all the drugs in the TI solution. A consequence of this delayed systemic absorption is that the local drug effect of a given dose of drug D is prolonged far beyond that which can be achieved by an equal dose IV or oral dose of D. Thus, the subcutaneous bioavailability of D, as determined by the area under the curve (AUC) of the concentration-time profile combined with the prolonged time within the tumescent therapeutic ambit range (TTAR), is often significantly greater by TI compared to IV, IM or oral delivery.

Slow systemic drug absorption associated with tumescent infiltration delivery of a highly dilute tumescent solution produces a serum concentration-time profile resembling a slow IV infusion with prolonged systemic effects. This slow “IV delivery” can replace or supplement the standard oral or IV delivery of the drug and results in less variation of serum drug concentrations.

An important pharmacokinetic advantage of TI drug delivery is a prolonged local drug effect (e.g., prolonged T>MIC) in relatively avascular subcutaneous fat and concomitant prolonged systemic concentrations with relatively small Cmax in serum. The small serum C. is particularly advantageous with tumescent infiltration antibiotic delivery to prevent or treat a localized skin infection while simultaneously minimizing the peak antibiotic concentration within the gut and thus reducing the risk of antibiotic-associated C. difficile diarrhea.

Large volume tumescent infiltration of dilute drug spreads drug throughout a larger area and larger volume of subcutaneous tissue than can be treated with injection of an equal mg dose at commercial out-of-the-bottle concentrations.

TI drug delivery can be performed by any primary care provider (physician, physician's assistant, nurse practitioner) in an office or clinic setting.

Examples of TI pharmacokinetic profiles include TI (antibiotic & lidocaine) delivery and TI (acyclovir & lidocaine) delivery:

1) Local and systemic TI antibiotic & lidocaine drug delivery prevents and treats systemic inflammatory response syndrome (SIRS) and bacterial sepsis. Briefly, local TI antibiotic & lidocaine delivery prevents SIRS by engulfing and isolating damaged (traumatized or infected) subcutaneous tissue within a persistent mass of vasoconstricted tumescent fluid. Systemic TI antibiotic & lidocaine delivery treats SIRS by significantly down regulating systemic inflammatory mediators. In addition, TI lidocaine delivery prevents platelet activation both locally and systemically and thereby and attenuates platelet-mediated inflammatory response. It is known that TI lidocaine prevents thromboembolism (U.S. Pat. No. 8,957,060 B2, Tumescent antibiotic solution).

2) Local and systemic TI acyclovir and lidocaine delivery for treatment of the acute pain and tissue damage of Herpes zoster dermatitis and prevention of dreaded post-herpetic neuralgia (PHN). It is known that tumescent lidocaine (without acyclovir) can relieve the pain of acute zoster dermatitis. With acyclovir in the tumescent solution, TI delivery achieves subcutaneous acyclovir concentrations that far exceed concentrations achievable by IV delivery; the result is decreased varicella zoster virus (VZV) replication, decreased extent, severity and of VZV dermatitis, shortened duration of VZV dermatitis, decreased inflammatory damage to nerves and decreased risk of chronic post-herpetic neuralgia. In addition, systemic absorption of acyclovir following subcutaneous TI acyclovir delivery produces sustained therapeutic serum acyclovir concentrations and thereby reduces VZV viremia. (A more detailed description of the therapeutic advantage of TI acyclovir delivery for treatment of the acute zoster dermatitis and preventing PHN is presented below).

TI allows direct subcutaneous infiltration of drugs which otherwise cannot be injected subcutaneously because of pain or tissue toxicity. Indeed, there are a number of drugs that are never injected subcutaneously because the Food and Drug Administration (FDA) approved package insert labeling states explicitly that the drug should NOT be injected subcutaneously.

Acyclovir is a specific example of a drug for which the FDA countermands subcutaneous injection. The FDA-approved package insert labeling for IV acyclovir states, “Acyclovir Injection is intended for intravenous infusion only, and should not be administered topically, intramuscularly, orally, subcutaneously, or in the eye.” Nevertheless, we have found that, in clinical practice, TI delivery of acyclovir is safe and effective.

Among the drugs that have been reported to be associated with subcutaneous tissue toxicity as the result of extravasation or infiltration at an IV site are the following: phenytoin, calcium gluconate, potassium chloride, calcium chloride, dopamine, dextrose solutions, epinephrine, sodium bicarbonate, nafcillin, propofol, norepinephrine, arginine, promethazine, vancomycin, tetracycline, dobutamine, vasopressin, acyclovir, amphotericin, ampicillin, cloxacillin, gentamicin, metronidazole, oxacillin, penicillin, amiodarone, albumin, furosemide, lorazepam, immunoglobulin, morphine, and sodium valproate. Careful formulation of dilute TI solutions of these drugs may allow safe and effective subcutaneous tumescent infiltration.

Pharmacologic properties that contribute to cutaneous and subcutaneous tissue toxicity include pH, osmolality, diluent, vasoactive properties, and inactive ingredients. With appropriate formulation of the subcutaneous TI solution these drugs can be injected (delivered) subcutaneously in a manner that is safe, comfortable and uniquely effective.

The possible safe and effective subcutaneous delivery of these drugs is unanticipated. The therapeutic advantage of TI (local and simultaneous systemic delivery) is not obvious. TI provides therapeutic subcutaneous concentrations that are not achievable by any other mode of delivery and TI simultaneously provides therapeutic serum concentrations with a pharmacokinetic concentration-time profile resembling a slow continuous IV infusion. No previously described mode of drug delivery can achieve these results.

Advantages to Using the Tumescent Technique

Tumescent delivery of pharmaceutical agents can provide a highly localized and sustained dosage of the pharmaceutical agent to the delivery site. For example, use of the tumescent technique to deliver Tumescent Local Antibiotics (TLAnti) can provide a high, sustained dosage of antibiotics directly to a surgical site. This has the advantage over the standard treatment with intravenous (IV) antibiotics in that the medication is concentrated and the dosage maximized at the area that is at risk of infection. In some embodiments, the concentration of the antibiotic drug and the local anesthetic drug within the TLAnti (which equals the maximum concentrations of these drugs within the tissues infiltrated with the TLAnti) far exceed the concentrations of these drugs which can be safely achieved by intravenous delivery. In embodiments of TLAnti comprising cefazolin, the concentration of antibiotic in the subcutaneous tissue at the surgical site may be three times or more than the measured maximum serum concentration of the same drug when administered intravenously prior to the procedure. Another advantage is that the therapeutic dosage of antibiotics at the surgical site lasts significantly longer with tumescent administration of TLAnti as compared to IV antibiotics. The result is that any bacteria present at the surgical site are exposed to a higher dosage of antibiotics for a longer period of time when TLAnti is used in place of IV antibiotics.

The bioavailability and effectiveness of an antibiotic can be assessed using the area under the curve (AUC) measurement of the tissue-concentration of the antibiotic as a function of time. After an IV infusion of an antibiotic, the serum-antibiotic AUC may be more than 100 times greater than the serum-antibiotic AUC following tumescent antibiotic delivery. On the other hand, the subcutaneous tissue-antibiotic AUC following the IV delivery of an antibiotic is less than 1/100th the tissue-antibiotic AUC following tumescent antibiotic delivery. Similarly, the peak serum concentrations of an antibiotic is higher after IV infusion compared to tumescent antibiotic, while the peak tissue concentration of antibiotic is lower after IV infusion compared to tumescent antibiotic. Tumescent antibiotic delivery produces significantly lower systemic concentrations of antibiotic while at the same time the local tissue concentration of antibiotic at the site of tumescent antibiotic infiltration is dramatically higher than that which can be achieved by IV antibiotic delivery.

Some embodiments relate to a method of using TLAnti during various surgical procedures. For example, in a liposuction procedure, a therapeutic quantity of TLAnti is injected into the subcutaneous compartment. Once sufficient anesthesia is achieved, another cannula is inserted and adipose tissue removed. The cannula is subsequently removed and the surgical site dressed and/or closed as appropriate. The high levels of antibiotics that remain for some period of time in the surgical site can reduce the risk of postoperative infection. Similarly, a large number of general surgical procedures including, but not limited to, open gastrointestinal surgery, obstetric surgery, orthopedic surgery, and vascular surgery are appropriate for the use of subcutaneous TAD.

The targeted application of highly concentrated antibiotics to the surgical site largely eliminates many of the problems inherent in systemic, prophylactic antibiotic use. In some embodiments where the tumescent solution comprises antibiotic and vasoconstrictive components, substantial quantities of antibiotics are injected into the surgical site using tumescent technique; however, the antibiotics enter the systemic circulation slowly due to the presence of a vasoconstrictive component. This delayed absorption minimizes the systemic antibiotic concentrations and reduces the possibility of the patient experiencing side effects compared with IV antibiotics. In addition, because the normal bacterial flora of the gut is not exposed to a bactericidal dosage of antibiotics when using the tumescent technique to deliver antibiotic, the risk of inadvertently eliminating the benign and protective bacteria in the gastrointestinal system is reduced. This reduces the likelihood of creating conditions favorable for the overgrowth of antibiotic-resistant and pathogenic bacteria such as Clostridium difficile. Finally, by not exposing any bacteria present beyond the surgical site to a therapeutic dosage of antibiotics, the risk of promoting the development of antibiotic resistant strains of a variety of pathogenic bacteria is minimized. This helps to reduce the problem of the spread of antibiotic resistant bacteria into the community.

Some embodiments relate to methods for using tumescent solutions in the subcutaneous space to treat a variety of medical conditions where systemic administration of medications is undesirable or impossible. Various embodiments include, but are not limited to, methods for using tumescent solutions as an anesthetic for medical procedures by clinicians, methods for using tumescent solutions in the administration of fluids to patients by medical professionals and first responders, methods for using tumescent antibiotic solutions to prevent and/or treat infections, methods for providing a chemotherapy agent to tissue after tumor removal and methods for using tumescent solutions in the controlled release of antibiotics and other pharmaceutical agents.

Tumescent administration of anesthetics, antibiotics, vasoconstrictors, and/or other pharmaceutical agents can improve the outcome of surgical procedures to remove tumors. Tumors may be benign or malignant, cancerous. Benign tumors are well circumscribed and are generally treated by surgery alone. Malignant/cancerous tumors on the other hand are more difficult to treat. When malignant tumors are localized, surgical removal is a common treatment option. Approximately 40% of all cancers are treated with surgery alone. In most other cases where surgery is an option, it is combined with other treatments—usually radiation therapy or chemotherapy. One danger of the surgical removal of malignant tumors is the possibility of spreading or seeding the cancerous cells during the process of removing the tumor. Tumescent delivery of a vasoconstrictor to the surgical site can reduce the risk of malignant cells entering the bloodstream. The tumescent technique may also be used to locally deliver chemotherapy agents. Local administration of chemotherapy agents allows for higher localized dosages of the chemotherapy agents than would be tolerated systemically and a reduction of adverse side effects. Examples of chemotherapy agents include, but are not limited to: actinomycin D, adriamycin, alkeran, ara-C, arsenic trioxide (trisenox), avastin, BiCNU, busulfan, carboplatinum, CCNU, cisplatinum, cytoxan, daunorubicin, DTIC, 5-FU, erlotinib, fludarabine, gemcitabine, herceptin, hydrea, idarubicin, ifosfamide, irinotecan, lapatinib, leustatin, 6-MP, methotrexate, mithramycin, mitomycin, mitoxantrone, navelbine, nitrogen mustard, rituxan, 6-TG, taxol, taxotere, topotecan, velban, vincristine, VP-16, and xeloda. Other anticancer drugs, such as angiogenesis inhibitors, may also be tumescently delivered. Examples of angiogenesis inhibitors include, but are not limited to, angiostatin, endostatin, and tumstatin.

In some embodiments, the tumescent solutions can be premixed and packaged prior to being sent to the provider. In other embodiments, one or more components of the tumescent solution can be added shortly before or during the medical procedure wherein they are to be used. In most embodiments, the bulk of the tumescent solution comprises a physiologically compatible solvent. Such solvents can include, for example, saline solution comprising sterile water and 0.9% sodium chloride. More dilute saline solutions can also be used. In other embodiments, a lactated Ringer's solution may be used. This comprises a mixture of sterile water, sodium, chloride, lactate, potassium and calcium that is isotonic with blood. Hartmann's solution can also be used as a solvent in some embodiments. Individuals skilled in the art will recognize that there are a wide variety of possible biologically compatible solvents for use in the solution.

In some embodiments, tumescent solution may be provided as a kit. In some embodiments, the tumescent solution is TLAnti. In one embodiment, TLAnti can be pre-mixed at a manufacturing site and distributed to practitioners in a ready to use form. In such embodiments, the TLAnti can be packaged in a form that allows easy interface with a tumescent reservoir or pumping system. Such packaging can come in a variety of sizes; however typical kits would include one liter or more of tumescent solution. In other embodiments, the tumescent solution may require rehydration or dilution to an administrable concentration.

In one embodiment, a kit can comprise a one-liter solution of 0.9% normal saline, 500 mg of cefazolin, 500 mg lidocaine 2%, 1 mg epinephrine, 10 mEq bicarbonate. One of ordinary skill in the art would recognize that several variations in the concentration of lidocaine are possible depending on the intended clinical use. For example, embodiments comprising higher dosages of lidocaine, optionally buffered with additional bicarbonate, can be used when a procedure is to be performed in a sensitive area. Variations on the type and concentration of antibiotic component are also possible. Some embodiments can also include various concentrations of epinephrine or different types of vasoconstrictors. Persons skilled in the art will recognize that many standardized variations are possible and the above example should not be deemed to be limiting.

In some embodiments, the tumescent solution or components for preparing the tumescent solution can be packaged along with a set of a cannula, tubing and possibly other surgical instruments for performing liposuction. Such kits can include an appropriate mix of tumescent solution components for the body part where the procedure is to be performed along with appropriately sized, sterile instruments. In some embodiments, the sterile instruments are capable of interacting with standardized liposuction equipment (i.e., peristaltic pumps, adipose tissue receptacles, etc.). Kits for TLAnti use in mastectomy procedures can be prepared comprising the tumescent solution along with any appropriate instruments.

In some embodiments, the tumescent solution can be provided in prefilled tumescent reservoir bags. Such bags could be manufactured by a pharmaceutical company and be sold as “ready to use.” Manufactured tumescent delivery bags are a more efficient and economical use of hospital staff than having to custom mix the tumescent solution for each surgical patient. Further, commercially produced prefilled tumescent reservoir bags would eliminate pharmacist error in mixing and preparing tumescent solution. In one embodiment, a TLAnti solution is provided in a prefilled tumescent reservoir bag comprising a dilute solution of local antibiotic such as lidocaine (≤1 g/L) or other water-soluble antibiotic and a vasoconstrictor, such as epinephrine (≤1 mg/L) in a physiologic electrolyte solution sodium chloride. TLAnti solutions containing epinephrine can be manufactured at a moderately acidic pH to optimize epinephrine stability. The TLAnti solution can be neutralized prior to administration by the addition of approximately 10-25 mEq of sodium bicarbonate. An appropriate amount of sodium bicarbonate can be included for addition to the prefilled tumescent reservoir bag.

Although TLAnti solution is safe when infiltrated into subcutaneous tissue; rapid, systemic infusion of TLAnti may be lethal. There is thus a need to prevent inadvertent IV administration of tumescent solutions. Various safety features may be incorporated into the prefilled tumescent reservoir bags. Tumescent reservoir bags can be designed to be readily distinguishable from standard IV bags. Distinguishing features include, but are not limited to, unique shape, color-coding, and/or printed warnings. In some embodiments, tumescent reservoir bags may be provided as kits in conjunction with a non-standard (non-luer) connector system to prevent inadvertent connection to an IV line.

Tumescent solution can be injected into the subcutaneous space during surgical procedures using a variety of infiltration cannula that are well known to persons skilled in performing surgical procedures. In some embodiments, the TLAnti can be injected into the treatment area using an infiltration cannula comprising a flexible cannula, a hub, and a rigid stylet. The flexible cannula has a proximal end and a distal end. The flexible cannula can also have a plurality of apertures disposed in a pattern about the distal end. The apertures are configured to infiltrate fluid into the subcutaneous tissue of a patient. The hub is configured to be held by a person performing the infiltration procedure. The hub has a first end and an opposing second end. The first end is attached to the proximal end of the flexible cannula and the second end includes a connector configured to connect to an input source for receiving the fluid to be infiltrated into the subcutaneous tissue of the patient. The fluid flows from the connector, through the hub and into the flexible cannula.

Several embodiments relate to a solution comprising an antibiotic component, an anesthetic component, a vasoconstrictor component and a solvent (hereafter referred to as Tumescent Local Antibiotics or TLAnti) to be delivered utilizing the tumescent technique. The relative concentrations of the components of TLAnti may be varied depending upon the level of anesthesia required at a given surgical site, the likelihood of bleeding, risk of infection, or other factors specific to the patient such as age, weight, or liver function.

In some embodiments, the anesthetic component may be comprised of a mixture of 2 or more anesthetics. In some embodiments, the vasoconstrictive component may be comprised of a mixture of 2 or more vasoconstrictors. In some embodiments, the antibiotic component may be comprised of a mixture of 2 or more antibiotics. In some embodiments the anesthetic component may possess both anesthetic and antibiotic properties. In some embodiments, TLAnti may additionally comprise an antiviral and/or an antifungal component. In some embodiments, the TLAnti may comprise additional pharmacological agents, such as, but not limited to anticonvulsants, stimulants, sedatives, antihistamines, retinoids, corticosteroids, calcium antagonists, chemotherapy agents, prostacyclins, and vasodilators.

In some embodiments, TLAnti comprises a water-soluble antibiotic component. In one embodiment, the water-soluble antibiotic may be Cefazolin. Cefazolin is a first-generation cephalosporin that has been sold under the brand names Ancef and Kefzol. This medication is particularly effective against many varieties of gram-positive bacteria that are typically present on the epidermal surface such as Staphylococcus aureus. Antibiotic coverage for such ubiquitous organisms is particularly important in surgical procedures because they can enter the surgical site during the procedure and are therefore a likely cause of post-operative infection. In some embodiments, cefazolin is used at a dosage of approximately 250 to 750 mg per liter of solvent. For example, in one embodiment 500 mg of cefazolin is used in 1 liter of TLAnti. In other embodiments cefazolin may be used at a dosage of approximately 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, or 900 mg per liter of solvent.

Persons skilled in the art will recognize that there are a variety of water-soluble antibiotics other than cefazolin that can be used in TLAnti. In some embodiments, TLAnti may comprise a combination of two or more water-soluble antibiotics. In some embodiments, penicillins, cephalosporins, carbapenems, aminoglycosides, sulfonamides, quinolones, macrolides, tetracyclines, lipopetides and oxazolidinones may be used. In one embodiment, metronidazole is used in TLAnti. Suitable antibiotics can be substituted in cases wherein a patient has a known or suspected hypersensitivity to a class of antibiotics, such as cephalosporins, or if the procedure is being performed in an area where resistance to a particular antibiotic is prevalent. In some embodiments, TLAnti may be used to treat an existing infection. In such embodiments, the infective agent may be determined and tested for antibiotic resistance. The antibiotic or combination of antibiotics may be specifically selected based on the resistance profile of the bacterial flora.

Examples of suitable antibiotics include, but are not limited to: amoxicillin, ampicillin, bacampicillin, carbenicillin, cloxacillin, dicloxacillin, flucloxacillin, mezlocillin, nafcillin, oxacillin, penicillin G, penicillin V, Piperacillin, Pivampicillin, Pivmecillinam, Ticarcillin, cefacetrile, cefadroxil, cephalexin, cefaloglycin, cefalonium, cefaloridine, cefalotin, cefamandole, cefapirin, cefatoxin, cefatrizine, cefazaflur, cephalexin, cefazedone, cefazolin, cefepime, cefradine, cefroxadine, ceftezole, cefaclor, cefonicid, cefprozil, cefuroxime, cefuzonam, cefmetazole, cefotetan, cefoxitin, loracarbef, cefbuperazone, cefmetazole, cefminox, cefotetan, cefoxitin, cefcapene, cefdaloxime, cefdinir, cefditoren, cefetamet, cefixime, cefmenoxime, cefodizime, cefotaxime, cefovecin, cefpimizole, cefpodoxime, cefteram, ceftibuten, ceftiofur, ceftiolene, ceftizoxime, ceftriaxone, cefoperazone, ceftazidime, latamoxef, cefclidine, cefepime, cefluprenam, cefoselis, cefozopran, cefpirome, cefquinome, flomoxef, ceftobiprole, ceftaroline, imipenem, meropenem, ertapenem, doripenem, panipenem, betamipron, biapenem, razupenem, amikacin, arbekacin, gentamicin, kanamycin, neomycin, netilmicin, paromomycin, rhodostreptomycin, streptomycin, tobramycin, apramycin, framycetin, ribostamycin, bekanamycin, dibekacin, tobramycin, spectinomycin, hygromycin B, paromomycin sulfate, sisomicin, isepamicin, verdamicin, astromicin, sulfasalazine, sulfamethoxazole, sulfamethizole, sulfisoxazole, fluoroquinolone, ketolide, ceftobiprole, flumequine, nalidixic acid, oxolinic acid, piromidic acid, pipemidic acid, rosoxacin, ciprofloxacin, enoxacin, lomefloxacin, nadifloxacin, norfloxacin, pefloxacin, rufloxacin, balofloxacin, gatifloxacin, grepafloxacin, levofloxacin, moxifloxacin, pazufloxacin, sparfloxacin, temafloxacin, tosufloxacin, clinafloxacin, gemifloxacin, sitafloxacin, trovafloxacin, prulifloxacin, azithromycin, erythromycin, clarithromycin, dirithromycin, roxithromycin, telithromycin, demeclocycline, doxycycline, minocycline, oxytetracycline, tetracycline, linezolid, clindamycin, metronidazole, vancomycin, rifabutin, rifampin, nitrofurantoin, chloramphenicol.

In several embodiments, TLAnti may also comprise an anesthetic component. In some embodiments, the anesthetic component may comprise lidocaine. In some embodiments, lidocaine may be provided at a concentration of between 30 mg and 1500 mg per liter of solvent. In some embodiments, lidocaine may be provided at a concentration of between 400 mg and 1250 mg per liter of solvent. In other embodiments, lidocaine may be provided at concentrations of 30 mg to 40 mg, 40 mg to 50 mg, 50 mg to 60 mg, 60 mg to 70 mg, 70 mg to 80 mg, 80 mg to 90 mg, 90 mg to 100 mg, 100 mg to 200 mg, 200 mg to 300 mg, 300 mg to 400 mg, 400 mg to 500 mg, 500 mg to 600 mg, 600 mg to 700 mg, 700 mg to 800 mg, 800 mg to 900 mg, 900 mg to 1,000 mg, 1,000 mg to 1,100 mg, 1,100 mg to 1,200 mg, 1,200 mg to 1,300 mg, 1,300 mg to 1,400 mg, 1,400 mg to 1,500 mg, and 500 mg to 1,000 mg per liter of solvent.

In some embodiments, anesthetics other than lidocaine can be used. Traditional local anesthetics include amide-type or ester-type local anesthetics. Non-traditional anesthetics include neurotoxin-based local anesthetics. Examples of anesthetics that are used in tumescent compositions include, but are not limited to saxitoxin, tetrodotoxin, benzocaine, chloroprocaine, cocaine, cyclomethycaine, dimethocaine, larocaine, propoxycaine, novocaine, proparacaine, tetracaine, amethocaine, articane, bupivacaine, carticaine, cinchocaine, dibucaine, etidocaine, levobupivacaine, mepivacaine, piperocaine, prilocaine, ropivacaine, trimecaine. In some embodiments, combinations of two or more anesthetics may be used. Suitable concentrations of anesthetic are approximately 30 mg to 40 mg, 40 mg to 50 mg, 50 mg to 60 mg, 60 mg to 70 mg, 70 mg to 80 mg, 80 mg to 90 mg, 90 mg to 100 mg, 100 mg to 200 mg, 200 mg to 300 mg, 300 mg to 400 mg, 400 mg to 500 mg, 500 mg to 600 mg, 600 mg to 700 mg, 700 mg to 800 mg, 800 mg to 900 mg, 900 mg to 1,000 mg, 1,000 mg to 1,100 mg, 1,100 mg to 1,200 mg, 1,200 mg to 1,300 mg, 1,300 mg to 1,400 mg, 1,400 mg to 1,500 mg, and 500 mg to 1,000 mg per liter of solvent.

The concentration of the anesthetic component can be varied depending on the sensitivity of the treatment area and the sensitivity of the patient to pain. If the TLAnti is to be used in sensitive areas such as the face or breasts, a higher concentration of anesthetic can be used. Lower concentrations of anesthetic can be used in the TLAnti solution for procedures in less-sensitive areas such as the hips.

Neurotoxins are a varied group of compounds, both chemically and pharmacologically. They vary in both chemical structure and mechanism of action and produce very distinct biological effects. Neurotoxins act on various ion channels, e.g., sodium, potassium, calcium and chloride channels. Neurotoxins acting on voltage-gated sodium channels can bind to six different sites in the channels, distinguished both by binding site(s) on the ion channels and by effect(s) of a toxin's action.

Non-traditional anesthetics or neurotoxin-based anesthetics may be administered by tumescent infiltration drug delivery. For instance, Neosaxitoxin (NeoSTX) is a site-1 sodium channel blocker that produces prolonged local anesthesia in animals and humans (Lobo, K. et al. 2015 Anesthesiology 123(4): 873-885). Neosaxitoxin can be used in small doses. Other neurotoxin-based local anesthetics include tetrodotoxin, saxitoxin and conotoxins, such as □-conotoxin (Prasanna, C. et al. (2014 Int J Anesthesiology Res 2: 11-15). In some embodiments, an anesthetic is efficacious for at least 6 hours, 12 hours, 18 hours, 24 hours, 30 hours, 36 hours or 48 hours. A local anesthetic solution that can last for 2 days is ideal for treating certain types of conditions, e.g., Herpes zoster (shingles).

In some embodiments, the tumescent solution can be delivered via a disposable catheter that can be used in emergency situations or under conditions when establishing intravenous access is difficult or impossible. In such embodiments, the tumescent solution can be injected into the subcutaneous space via a flexible cannula with a rigid stylet that can be fabricated from stainless metal or rigid plastic. The distal end of the cannula can be closed to cover the tip of the rigid stylet or open with a hole allowing the tip of the rigid stylet to protrude. In some embodiments, the tip of the rigid stylet can be sharp to facilitate the direct insertion through the skin of the patient. Other embodiments comprise a blunt tip requiring a skin incision to permit insertion of the rigid stylet and the cannula into the subcutaneous space. The stylet can be formed to have either a solid or hollow cross-sectional configuration. The hollow rigid stylet may have small holes distributed along its length in a pattern dissimilar or identical to the pattern of holes placed along the flexible cannula into which the stylet is inserted. Thus, in some embodiments, the stylet itself can be used as an infiltration cannula.

Our research has provided objective pharmacokinetic data that, in some embodiments, support approximately 28 mg/kg for infiltration local anesthesia. Compared to general anesthesia, Tumescent Infiltration Lidocaine Anesthesia has the ability to:
1) Improve patient safety (reduce the need for general anesthesia),
2) Improve patient comfort by reducing PONV (post-operative nausea and vomiting) reducing hypothermia and providing prolonged post-operative analgesia
3) Reduce cost,
4) Reduce the risk of post-operative venous thromboembolism (tumescent lidocaine significantly reduces systemic platelet activation without adversely affecting surgical hemostasis, manuscript in preparation).
5) Provide improved pre-emptive, intraoperative and postoperative anesthesia and analgesia and thus reduce the need for narcotic analgesia while accelerating earlier post-op ambulation
6) Reduce the risk of post-surgical neuropathic pain by reducing inflammation and neurologic peripheral and central sensitization.
7) Reduce the risk of excessive systemic inflammatory response (systemic lidocaine attenuates activity of inflammatory mediator associated with innate immunity and thus reduce the risk of sepsis and systemic inflammatory response syndrome.
8) Reduce the risk of surgical site infections (SSI) by subcutaneous infiltration of tumescent solution containing antibiotic(s), e.g., cefazolin and metronidazole.
9) Completely eliminate the pain and reduce the duration of acute Herpes zoster dermatitis and reduce the risk of chronic post-herpetic neuralgia (PHN) by tumescent infiltration of a TLA solution containing a dilute solution of acyclovir.

In contrast to the tumescent infiltration technique disclosed herein, which allows for relatively high drug dosages, including for lidocaine, continued FDA insistence on a lower maximum lidocaine dosage of 7 mg/kg by conventional methods presents a major impediment to improvement in patient care and a dramatic reduction in the cost of care.

Riboflavin (vitamin B2) and Topical Irrigation Solutions

Another application of B12 or B2 as color-indicators for a drug solution in an IV bag concerns the treatment of chronic wounds by frequent cleansing and irrigation assisted by use of a tumescent peristaltic roller pump.

The worldwide costs of chronic wound care are enormous. Among individuals over 65 years in the United States the prevalence of having a wound at any one time, is approximately 3% (Reiber G. E. Diabetic foot care. Financial implications and practice guidelines. Diabetes Care. 1992; 15 (Suppl. 1): 29-31) with an estimated annual cost of approximately US $25 billion (Sen C. K., Gordillo G. M., Roy S., Kirsner R., Lambert L., Hunt T. K., Gottrup F., Gurtner G. C., Longaker M. T. Human skin wounds: A major and snowballing threat to public health and the economy. Wound Repair Regen. 2009; 17:763-771). Techniques for clinical wound care are evolving.

Chronic wounds are complicated by biofilm infections. Biofilm infections do not readily respond to systemic antibiotics.

It is our clinical experience that biofilms do respond to fastidious regular wound cleansing and irrigation repeated approximately four times daily. This improved success in treating stubborn chronic wound infections can be achieved by wound irrigation using a peristaltic roller pump and pump tubing to produce a moderately forceful stream of dilute hypochlorous acid (HOCl) at concentrations that range between 0.1% and 0.001%.

Irrigation solutions that are delivered into an open wound by a peristaltic pump, are typically contained in an IV bag. When wound irrigation is assisted by use of a peristaltic roller pump, the irrigating solution containing HOCl acid is most conveniently prepared in an IV bag of saline which can be connected to a peristaltic roller pump and tubing to forcefully pump the irrigation solution onto and into the open wound in such a way that the hydrodynamic force of the stream of fluid removes necrotic debris and efficiently delivers the HOCl solution onto the wound surface and into its interstices.

IV injection of HOCl acid may be neither safe nor effective. To reduce the risk of inadvertent IV injection of HOCl, riboflavin (vitamin B2), as well as B12, can be added to the irrigation solution as a color-indicator in the IV bag containing HOCl as warning against IV infusion. The contents of an irrigation solution, such as HOCl, should not be injected either IV or subcutaneously. It is proposed that the addition of yellow-colored riboflavin to an IV bag can provide a warning that the fluid should neither be injected IV nor subcutaneously.

The treatment of chronic open wounds is especially challenging. Decubitus ulcers (pressure sores) are among the most difficult of all open wounds to treat successfully. Decubitus wounds are typified by tissue hypoxia resulting from chronic inflammation induced by biofilm infection. A novel therapeutic approach involves irrigation of decubitus ulcers with forceful stream of antiseptic solution provided by tumescent peristaltic infiltration pump that disrupts the biofilm by the impact of fluid flow through an open-ended small gauge cannula. The antiseptic solution is safe for topical application onto the surface of the wound but should never be injected into tissue or infused intravenously. The antiseptic irrigation solution is typically prepared in an IV bag for delivery to the wound with the assistance of a peristaltic pump.

The topical 0.02% hypochlorous acid (HOCl) antiseptic solution is a safe, over the counter (OTC) non-toxic antimicrobial when applied to the skin or an open wound. However, 0.02% hypochlorous acid is not approved for intravenous or injection into tissue.

The realization that putting B12 into a bag of TEL is a nonobvious answer to a problem that is largely unrecognized. B12/B2 provide uniquely visible warnings to anyone that the IV bag contains something that is potentially hazardous as well as unexpected to be found in an IV bag of saline. The color-indicator can be seen at a distance by anyone present in the room, including the patient, as well as the clinical staff. Further, the color-indicator is independent of any written language. This is a distinct advantage over a written warning-label.

TELB consists of epinephrine (1 mg/L)+lidocaine (1 gm/L)+either B12 (cyanocobalamin 10 mg/L) or vitamin B2 (riboflavin 100 mg/L) in 0.9% physiologic normal saline (NS). In an aqueous solution the color of B12 is pink and B2 is yellow. B12 and B2 have three important functions:
1) TELB functions as an excellent excipient solution for tumescent drug delivery.
2) B12 and B2 improve wound healing.
3) B12 seems to have a beneficial effect in treating Herpes zoster.

Bags containing TEL are commonly prepared for use during mastectomy, breast reduction, abdominoplasty, and liposuction. It is not credible that such bags will never be inadvertently infused intravenously. Indeed, I know of at least one fatal case of unsuspected intravenous delivery of TEL. To the best of my knowledge, tumescent epinephrine lidocaine, AKA tumescent local anesthesia (TLA), is the only drug that is deliberately placed in IV bags and specifically NOT intended for IV delivery. After all, who would expect that a pharmacist would intentionally put a drug into an IV bag if the IV infusion of that drug might be lethal? Who would expect that a responsible surgeon would allow a TEL bag to be taken to an operating room where there are likely to be multiple, nearly identical appearing IV bags specifically intended for IV delivery?

There is a need for a non-toxic color-indicator for solutions in an IV bag, which should never be given intravenously. The use of vitamin B12 and vitamin B2 as color-indicators to improve patient safety was nonobvious.

Methods of Treating a Localized Viral Infection

The tumescent technique may be used to treat localized viral infections. Surprisingly, while some antibiotics and antiviral agents are not recommended for subcutaneous use, the tumescent technique allows such agents to be safely used at relatively high, localized therapeutic dosages. For instance, the antibiotic gentamycin and the antiviral compound Acyclovir, which is commonly used to treat infections caused by herpes viruses, such as genital herpes, cold sores, shingles, and chicken pox, are not recommended for subcutaneous administration. With the tumescent technique, there is a relatively large total amount of drug in the patient's body, but the drug is isolated from the systemic circulation because only the drug on the outer boundary of the mass of drug is the available for absorption, whereas the portion of the drug located within the central portion of the mass of fluid is virtually isolated from the systemic circulation by virtue of profound capillary vasoconstriction.

The tumescent technique is amenable to treatment of localized viral infection, such as for treatment of viral diseases related to herpes virus (including Herpes Simplex I, Herpes Simplex II, herpes zoster (shingles), herpetic conjunctivitis, keratitis, and genital herpes). Other types of localized viral infection include Molluscum Contagiosum, a common skin infection caused by a pox virus that affects both children and adults and Kaposi's sarcoma (KS), a connective tissue cancer caused by human herpes virus 8.

A non-exhaustive list of antiviral agents used to treat localized viral infection, e.g., shingles, includes: Abacavir, Acyclovir, Adefovir, Amantadine, Amprenavir, Ampligen, Arbidol, Atazanavir, Atripla, Balavir, Brivudine, Cidofovir, Combivir, Dolutegravir, Darunavir, Delavirdine, Didanosine, Docosanol, Edoxudine, Efavirenz, Emtricitabine, Enfuvirtide, Entecavir, Ecoliever, Famciclovir, Fomivirsen, Fosamprenavir, Foscarnet, Fosfonet, Ganciclovir, Ibacitabine, Imunovir, Idoxuridine, Imiquimod, Interferon type I, Interferon, Lamivudine, Lopinavir, Loviride, Maraviroc, Moroxydine, Methisazone, Nelfinavir, Nevirapine, Nexavir, Nucleoside analogues, Novir, Oseltamivir, Peginterferon alfa-2a, Penciclovir, Peramivir, Pleconaril, Raltegravir, Ribavirin, Rimantadine, Ritonavir, Pyramidine, Saquinavir, Sofosbuvir, Telaprevir, Tenofovir, Tenofovir disoproxil, Tipranavir, Trifluridine, Trizivir, Tromantadine, Truvada, Valaciclovir, Valganciclovir, Vicriviroc, Vidarabine, Viramidine, Zalcitabine, Zanamivir, Zidovudine.

In tumescent antiviral drug delivery, the subcutaneous concentration of the antiviral agent achieved is simultaneously: (i) below the threshold for local tissue toxicity while sufficiently concentrated to result in a significant positive local therapeutic effect, and (ii) greater than the maximum subcutaneous interstitial fluid concentration that can be achieved by conventional intravenous delivery or oral delivery of the antiviral agent. For example the subcutaneous concentration of the antiviral agent achieved is equal to or about 5%, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, 200%, 250%, 300%, 350%, 400%, 450% or 500% greater than the maximum subcutaneous interstitial fluid concentration that can be achieved by conventional intravenous delivery or oral delivery of the antiviral agent.

In some embodiments, Acyclovir is used at a diluted concentration of 0.1 g/L-10 g/L, preferably 0.5 g/L-5 g/L, more preferably 1-2 g/L, or at 1 g/L.

In some embodiments, Gentamycin is used at a diluted concentration of 0.1 mg/L-1 g/L, preferably 50-800 mg/L, more preferably 50-200 mg/L or at 80 mg/L.

A tumescent composition used to treat localized viral infection typically contains an antiviral component and a vasoconstrictor. The tumescent composition may optionally comprise other components, such as antibiotic, anesthetic and anti-inflammatory components.

Tumescent Drug Delivery of an Antiviral Agent for Herpes Zoster

Pathophysiology of Herpes Zoster Dermatitis

Varicella-zoster virus (VZV) causes varicella (chickenpox) and herpes zoster (shingles). Varicella (chickenpox) is highly contagious. It initially enters the host by penetrating the respiratory epithelium causing viremia and the classic vesicular chickenpox rash. From the skin, VZV migrates within cutaneous sensory neurons to arrive at sensory dorsal root ganglia (DRG). As the host develops a cellular immunity involving CD4 & CD8 cells and a serologic immune response, the rash subsides and the VZV virus within the ganglion becomes latent.

Years later, with age-related waning of cellular immunity or with immunodeficiency (chemotherapy or HIV infection) the latent VZV can reactivate, proliferate and migrate along a sensory nerve from the dorsal root ganglion toward the skin of the corresponding dermatome.

The initial manifestation of H zoster is abrupt onset of localized pain that extends over the next few days within a localized unilateral area of skin spanning 1 to 3 adjacent dermatomes. Patients often attribute the acute onset of the pain to be the result of muscle strain, back-muscle spasm, or a bacterial infection. Within 2-4 days there is the onset of The host's immune response produces an intense inflammatory reaction with a potential for permanent sensory nerve injury.

The area and intensity of painful skin tends to enlarge over the next 2 to 3 days. Zoster pain is intense and has 3 clinically distinct components: a deep burning unremitting pain, sporadic acute sharp lancinating pain, and a paresthetic pain that is elicited by light touch or temperature change (mechanical allodynia).

Within 2 to 3 days after the onset of pain, there is the appearance of a characteristic erythematous papulovesicular rash. Soon the vesicles enlarge and coalesce into bullae distributed over the affected dermatome(s). The vesiculation and bulla formation progress to epidermal crusting and necrosis, which then resolves over 14 days or so. Usually, the pain of zoster gradually resolves over several weeks to months.

Post Herpetic Neuralgia (PHN)

Post herpetic neuralgia (PHN) is the most common and perhaps the most dreaded complication of Herpes zoster. PHN is defined as pain that persists for more than 3 months after acute herpes zoster. PHN often does not respond well to narcotics or other analgesics. The incidence of post herpetic neuralgia (PHN) is approximately 10%, but among patients with hematologic malignancy it is at least 48%.

Herpes zoster patients with greater pain and rash severity have greater risk of PHN. This suggests that greater neural damage (caused by more severe acute infection) contributes to risk of PHN. Indeed, acute pain severity is a major risk factor for PHN (Dworkin R H, Boon R J, Griffin D R G. Postherpetic neuralgia: Impact of Famciclovir, age, rash severity and acute pain in Herpes zoster patients. JID 1998; 178 (Supp 1) S76-S80). Current approaches to acute zoster pain rely on aggressive analgesic intervention that merely attenuates zoster pain. Only TI acyclovir (tumescent lidocaine and acyclovir) is capable of eliminating 100% of acute zoster pain for up to 12 to 18 hours and providing unprecedented high tissue concentrations, superior bioavailability within affected cutaneous tissue and prolonged time within the tumescent therapeutic ambit range (TTAR) of acyclovir.

Because PHN is a neuropathic pain that is resistant to treatment, preventing PHN is of prime importance. TI of acyclovir delivers unprecedented high and prolonged subcutaneous concentrations of lidocaine, eliminates 100% of pain for up to 12 hours or more without repeat dosing and thus reduces the risk of neuropathic pain and PHN. In this fashion Tumescent lidocaine+acyclovir is more effective than IV acyclovir at reducing the risk of PHN.

It is known that the risk of developing H zoster is closely correlated with a person's age and immune competence in suppressing VZV proliferation. However, once the H zoster occurs the pathogenesis of PHN is not completely understood.

The zoster blister contains large amounts of infectious VZV viral particles. The risk of severe H. zoster and the risk of PHN are closely correlated with the intensity of the host's secondary inflammatory immune response to VZV, the degree of pathologic damage to sensory nerves, the total area of blistering, the intensity and duration of epidermal necrosis, and the intensity and duration of acute pain.

Early treatment with oral anti-VZV drugs or IV acyclovir shortens the duration and intensity of acute zoster pain. Higher doses of famciclovir have been shown to reduce the duration of zoster pain and the risk of PHN. However, the bioavailability of systemic delivery of anti-VZV drugs within zoster-affected subcutaneous tissue is limited by cutaneous edema and capillary necrosis. Results of our pharmacokinetic research (our unpublished data) show that tumescent infiltration of a drug dramatically increases drug bioavailability in cutaneous interstitial fluid by one to two orders of magnitude.

Antiviral drugs do not provide anesthesia and do not eliminate acute zoster pain. The intensity and duration of any acute pain increases the risk of a permanent neuropathic pain syndrome. PHN is an example of neuropathic pain. Acyclovir, valacyclovir and famciclovir treat certain, but not all, aspects of H. zoster. They reduce the intensity and duration of zoster pain and decrease the risk of developing PHN. Only Tumescent lidocaine+acyclovir can both eliminate 100% of pain (for hours) and can decrease viral replication rate and the extent and intensity. The risk factors that have been shown to increase the incidence of PHN include: a) increasing age, b) intensity of pain upon initial presentation, c) duration of pain upon initial presentation, d) extent of the H. zoster rash upon presentation, e) intensity of pain one week after initiating antiviral therapy, f) progression of pain one week after initiating antiviral therapy and g) progression of dermatitis one week after initiating antiviral therapy.

An important embodiment of the present invention is the safe and effective subcutaneous infiltration of an antiviral agent for the treatment of Herpes zoster. Herpes Zoster or Shingles is an unusually painful disease caused by the varicella zoster virus that affects one million people in the United States annually. Among persons 85 years of age or older, 50% will eventually have herpes zoster. H. zoster can progress into chronic, potentially devastating, post-herpetic neuralgia (PHN).

Methods disclosed herein involve the subcutaneous infiltration of a tumescent solution of dilute lidocaine and epinephrine and one or more zoster-specific antiviral drugs (e.g., acyclovir) and/or a broad-spectrum antiviral (e.g., cidofovir), with or without anti-inflammatory drugs (e.g., steroidal anti-inflammatory drugs such as triamcinolone, non-steroidal anti-inflammatories)), and with or without sodium bicarbonate.

The beneficial result of subcutaneous delivery of acyclovir by tumescent infiltration (TI) to treat shingles is unexpected and not obvious. The FDA-approved package insert for ZOVIRAX® (5%=1 gm/20 ml) (acyclovir sodium) for Injection states, “WARNINGS: ZOVIRAX for Injection is intended for intravenous infusion only, and should not be administered topically, intramuscularly, orally, subcutaneously, or in the eye.” As disclosed herein, inclusion of a non-toxic compound that imparts a visible color to the solution, e.g., Vitamin B12 and Vitamin B2, serves as a color warning to medical practitioners to not use a tumescent solution intravenously.

There are no published accounts of the clinical use of subcutaneously injected acyclovir in human subjects. Thus, tumescent infiltration of acyclovir is unexpected and not obvious. Extravasated acyclovir is a known complication of IV delivery and can produce significant inflammation, bullae and pain.

In contrast to the teachings of those skilled in the art of treating patients with zoster, we have found that a subcutaneous infiltration of dilute solution of an antiviral agent, such as acyclovir, in a tumescent solution is remarkably safe and effective at
1) eliminating acute zoster pain for at least 12 hours or more,
2) attenuating the progression of blistering zoster dermatitis and
3) reducing the risk of developing PHN.

The antiviral drugs acyclovir (Zovirax®), valacyclovir (Valtrex®) and famcyclovir (Famvir®) effectively treat H. zoster. Only Acyclovir is available for IV delivery. At present, oral delivery is considered sufficient for most cases of H. zoster. IV delivery is usually reserved for patients requiring hospitalization, for example disseminated H. zoster in immunocompromised hematopoetic transplant patients or HIV (AIDS) patients, severe forms of zoster such as herpes zoster ophthalmicus that can cause blindness, herpes oticus (Ramsay-Hunt syndrome) which can cause unilateral facial paralysis and/or permanent hearing loss, CNS zoster and Zoster pneumonia. In general, the sooner treatment begins, the less severe the intensity of the rash and pain and the shorter its duration.

Subcutaneous tumescent drug delivery of acyclovir and lidocaine (TAD-acyclovir) typically eliminates 100% of acute zoster pain for 12 hours or more. This is unique among all forms of Herpes zoster treatments. Using an elastomeric pump to provide continuous subcutaneous infiltration, TI-acyclovir can eliminate zoster pain for days.

Greater subcutaneous acyclovir bioavailability and concentrations:
1) decrease VZV replication/proliferation within the dermatomal skin and nerves,
2) decrease VZV-induced inflammation,
3) decrease damage to skin and decrease local sensory nerve damage,
4) reduce the intensity, extent and duration of acute pain,
5) reduce the likelihood of developing chronic post herpetic neuralgia, and
6) reduce post-zoster hypopigmentation and scaring.

Tumescent Infiltration Antifungal Delivery

Tumescent infiltration can also be used to treat local and systemic fungal infections. A partial list of antifungal drugs includes:
Polyenes: amphotericin B, candicidin, Filipin, Hamycin, Natamycin, Nystatin and Rimocidin;
Imidazoles: Bifonazole, Butoconazole, Clotrimazole, Econazole, Fenticonazole, Isoconazole, Ketoconazole, Luliconazole, Miconazole, Omoconazole, Oxiconazole, Sertaconazole, Sulconazole and Tioconazole;
Triazoles: Albaconazole, Efinaconazole, Epoxiconazole, Fluconazole, Isavuconazole, Itraconazole, Posaconazole, Propiconazole, Ravuconazole, Terconazole and Voriconazole; and
Thiazoles: Abafungin.

Tumescent Infiltration Antiprotozoal Delivery

Tumescent infiltration can also be used to treat local and systemic protozoa infections. A partial list of antiprotozoal drugs includes:
Antinematodes: Mebendazole, Pyrantel pamoate, Thiabendazole, Diethylcarbamazine, and Ivermectin;
Anticestodes: Niclosamide, Praziquantel, and Albendazole;
Antitrematodes: Praziquantel;
Antiamoebics: Rifampin and Amphotericin B; and
Antiprotozoals: Melarsoprol, Eflornithine, Metronidazole, Tinidazole and Miltefosine.

Methods of Treating Localized Neuropathic Pain

Neuropathic pain is a complex, chronic pain state that is generally accompanied by tissue injury. With neuropathic pain, the nerve fibers themselves might be damaged, dysfunctional, or injured. These damaged nerve fibers send incorrect signals to other pain centers. The clinical causes of neuropathic pain are diverse and include both trauma and disease. For example, traumatic nerve compression or crush and traumatic injury to the brain or spinal cord are common causes of neuropathic pain. Furthermore, most traumatic nerve injuries also cause the formation of neuromas, in which pain occurs as a result of aberrant nerve regeneration. In addition, cancer-related neuropathic pain is caused when tumor growth painfully compresses adjacent nerves, brain or spinal cord. Neuropathic pain can be caused by various diseases, such as viral infections and diabetes and alcoholism. For example, post herpetic neuralgia is caused by herpes viral infection and can cause moderate to severe chronic pain in the infected skin area to the subject.

Unfortunately, the available drug therapies for neuropathic pain often do not provide adequate pain relief. In addition, current therapies have serious side-effects including, for example, cognitive changes, sedation, nausea and, in the case of narcotic drugs, addiction. Many patients suffering from neuropathic pain are elderly or have other medical conditions that particularly limit their tolerance of the side-effects associated with available drug therapy. A number of anti-inflammatory, anxiolytic, narcotic and even anti-convulsants are currently used by the practitioners to treat neuropathic pain, but with limited success.

Selective serotonin reuptake inhibitors (SSRIs like paroxetine and citalopram) and other antidepressants (venlafaxine, bupropion) have been used in some patients.

Another common treatment of neuropathic pain includes anti-seizure medications (carbamazepine, phenytoin, gabapentin, lamotrigine, and others). Pregabalin and duloxetine can also be effective for nerve pain. Like amitriptyline, they may be given alongside other pain medications in the most troublesome nerve pain conditions. Duloxetine is licensed for pain from nerve damage resulting from diabetes, which most often starts in the feet.

The inadequacy of current therapy in relieving neuropathic pain calls for new compositions and methodologies of addressing the physical and social needs of the patient suffering from such condition. Methods of alleviating neuropathic pain would improve the quality of life for many people suffering from pain due to trauma or disease.

Neuropathic pain may be brought on by trauma, disease or irritation. There are countless types of neuropathic pain. Some of the common types include:
Postherpetic neuralgia. Postherpetic neuralgia is neuropathic pain that is brought on by an outbreak of shingles and persists after the condition has cleared.
Trigeminal neuralgia. Trigeminal neuralgia is characterized by shooting neck and facial pain. The pain is often worse with light touch and may make activities like shaving very painful.
Phantom limb pain. Phantom limb pain can occur in some people after a limb is amputated. This pain feels as if it is coming from part of the limb that is no longer there.
Diabetic neuropathy. Diabetic neuropathy causes burning or stabbing pain in the hands and feet of some people who suffer from diabetes.
Carpal tunnel syndrome. Carpal tunnel syndrome is caused by nerve compression in the wrists, and causes pain in the wrist, thumb and fingers.
Sciatica. Sciatica is caused by compression or irritation of the sciatic nerve, and often results in shooting pain that radiates down the back of leg.

Chronic neuropathic pain can also be caused by other chronic pain disorders. For instance, someone with degenerative disk disease, a form of arthritis, may experience neuropathic back pain if the condition causes damage to the nerves entering or exiting the spine. Some other conditions that may cause chronic neuropathic pain include spinal cord injury, post-surgical pain and cancer.

In tumescent drug delivery for neuropathic pain, the subcutaneous concentration of drug achieved is simultaneously: (i) below the threshold for local tissue toxicity while sufficiently concentrated to result in a significant positive local therapeutic effect, and (ii) greater than the maximum subcutaneous interstitial fluid concentration that can be achieved by conventional intravenous delivery or oral delivery of the drug. For example the subcutaneous concentration of the drug achieved is equal to or about 5%, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, 200%, 250%, 300%, 350%, 400%, 450% or 500% greater than the maximum subcutaneous interstitial fluid concentration that can be achieved by conventional intravenous delivery or oral delivery of the drug.

Methods of Treating a Localized Inflammation

In cases of localized inflammation, the tumescent technique allows anti-inflammatory agents to be safely administered at relatively high concentrations. Examples of anti-inflammatories include but are not limited to glucocorticoids and non-steroidal anti-inflammatory drugs (NSAIDS). Persons skilled in the art will note that there are a number of potential compounds that can be added to the tumescent composition.

Example glucocorticoids include triamcinolone, dexamethsasone, prednisolone, methylprednisolone, budesonide betamethasone, hydrocortisone and cortisone.

Example NSAIDS include Aspirin (Anacin®, Ascriptin®, Bayer®, Bufferin®, Ecotrin®, Excedrin®); choline and magnesium salicylates (choline magnesium trisalicylate (CMT), Tricosal®, Trilisate®); Choline salicylate (Arthropan®); Celecoxib (Celebrex®); Diclofenac potassium (Cataflam®); Diclofenac sodium (Voltaren®, Voltaren XR®); Diclofenac sodium with misoprostol (Arthrotec®); Diflunisal (Dolobid®); Etodolac (Lodine®, Lodine XL®); Fenoprofen calcium (Nalfon®); Flurbiprofen (Ansaid®); Ibuprofen (Advil®, Motrin®, Motrin TB®, Nuprin®); Indomethacin (Indocin®, Indocin SRC)); Ketoprofen (Actron®, Orudis®, Orudis KT®, Oruvail); Magnesium salicylate (Arthritab®, Bayer Select®, Doan's Pills®, Magan®, Mobidin®, Mobogesic®); Meclofenamate sodium (Meclomen®); Mefenamic acid (Ponstel®); Meloxicam (Mobic®); Nabumetone (Relafen®); Naproxen (Naprosyn®, Naprelan); Naproxen sodium (Aleve®, Anaprox®); Oxaprozin (Daypro®); Piroxicam (Feldene®); Rofecoxib (Vioxx®); Salsalate (Amigesic®, Anaflex 750®, Disalcid®, Marthritic®, Mono-Gesic C), Salflex®, Salsitab®); Sodium salicylate (various generics); Sulindac (Clinoril®); Tolmetin sodium (Tolectin®) and Valdecoxib (Bextra®).

Natural and synthetic glucocorticoids remain at the forefront of anti-inflammatory and immunosuppressive therapies. They are widely used to treat both acute and chronic inflammations, including rheumatoid arthritis, inflammatory bowel disease, multiple sclerosis, psoriasis and eczema, as well as being used in treatment of certain leukaemias and in immunosuppressive regimes following organ transplant. The anti-inflammatory effects are mediated either by direct binding of the glucocorticoid/glucocorticoid receptor complex to glucocorticoid responsive elements in the promoter region of genes, or by an interaction of this complex with other transcription factors, in particular activating protein-1 or nuclear factor-kappaB. Glucocorticoids inhibit many inflammation-associated molecules such as cytokines, chemokines, arachidonic acid metabolites, and adhesion molecules.

NSAIDs comprise a large class of drugs with many different options. In addition to aspirin, there are currently several types of both non-prescription (over-the-counter) NSAIDs and prescription brands of NSAIDs. The three types of NSAIDs most commonly used to treat many types of back pain and neck pain include: Ibuprofen (e.g., brand names Advil®, Motrin®, Nuprin®); Naproxen (e.g., brand names Aleve®, Naprosyn®), and COX-2 inhibitors (e.g., Celebrex®).

In tumescent delivery of an anti-inflammatory drug, the subcutaneous concentration of the anti-inflammatory drug achieved is simultaneously: (i) below the threshold for local tissue toxicity while sufficiently concentrated to result in a significant positive local therapeutic effect, and (ii) greater than the maximum subcutaneous interstitial fluid concentration that can be achieved by conventional intravenous delivery or oral delivery of the anti-inflammatory drug. For example the subcutaneous concentration of the anti-inflammatory drug achieved is equal to or about 5%, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, 200%, 250%, 300%, 350%, 400%, 450% or 500% greater than the maximum subcutaneous interstitial fluid concentration that can be achieved by conventional intravenous delivery or oral delivery of the anti-inflammatory drug.

Methods of Treating a Localized Cancer

Treating a localized cancer or reducing the growth of a tumor by localized delivery of a cancer medication can be achieved by using the tumescent technique. Using chemotherapy to treat cancer typically has unpleasant side effects. The toxic effects of the medication affect healthy cells, as well as those of the tumor itself. This leads to symptoms like nausea, hair loss or reduced effectiveness of the immune system. The tumescent technique allows higher doses of medication to be used, while the rest of the patient's body remains unaffected.

Chemotherapy agents are selected based on the type of cancer, the stage of the cancer (how far it has spread), the patient's age, the patient's overall health, other serious health problems (such as heart, liver, or kidney diseases) and the types of cancer treatments given in the past.

Chemotherapy regimens or treatment plans may use a single drug or a combination of drugs, which may be more effective than a single drug, because the cancer cells can be attacked in several different ways.

Alkylating Agents

Alkylating agents directly damage DNA (the genetic material in each cell) to keep the cell from reproducing. These drugs work in all phases of the cell cycle and are used to treat many different cancers, including leukemia, lymphoma, Hodgkin disease, multiple myeloma, and sarcoma, as well as cancers of the lung, breast, and ovary. Because these drugs damage DNA, they can cause long-term damage to the bone marrow. In rare cases, this can lead to acute leukemia. The risk of leukemia from alkylating agents is “dose-dependent,” meaning that the risk is small with lower doses but goes up as the total amount of the drug used gets higher. The risk of leukemia after getting alkylating agents is highest about 5 to 10 years after treatment.

Alkylating agents are divided into different classes, including Nitrogen mustards: such as mechlorethamine (nitrogen mustard), chlorambucil, cyclophosphamide (Cytoxan®), ifosfamide, and melphalan; Nitrosoureas: such as streptozocin, carmustine (BCNU), and lomustine; Alkyl sulfonates: busulfan; Triazines: dacarbazine (DTIC) and temozolomide (Temodar®); and Ethylenimines: thiotepa and altretamine (hexamethylmelamine).

The platinum drugs (such as cisplatin, carboplatin, and oxalaplatin) are sometimes grouped with alkylating agents because they kill cells in a similar way. These drugs are less likely than the alkylating agents to cause leukemia later.

Antimetabolites

Antimetabolites interfere with DNA and RNA growth by substituting for the normal building blocks of RNA and DNA. These agents damage cells during the S phase, when the cell's chromosomes are being copied. They are commonly used to treat leukemias, cancers of the breast, ovary, and the intestinal tract, as well as other types of cancer.

Examples of antimetabolites include: 5-fluorouracil (5-FU), 6-mercaptopurine (6-MP), Capecitabine (Xeloda®), Cytarabine (Ara-C®), Floxuridine, Fludarabine, Gemcitabine (Gemzar®), Hydroxyurea, Methotrexate and Pemetrexed (Alimta®).

Anti-Tumor Antibiotics

These drugs are not like antibiotics used to treat infections. They work by altering the DNA inside cancer cells to keep them from growing and multiplying.

1. Anthracyclines

Anthracyclines are anti-tumor antibiotics that interfere with enzymes involved in DNA replication. These drugs work in all phases of the cell cycle. They are widely used for a variety of cancers. Examples of anthracyclines include: Daunorubicin, Doxorubicin (Adriamycin®), Epirubicin and Idarubicin. A major concern when giving these drugs systemically is that they can permanently damage the heart if given in high doses. For this reason, lifetime dose limits are often placed on these drugs. However, with the tumescent technique, this problem is avoided.

2. Other Anti-Tumor Antibiotics

Anti-tumor antibiotics that are not anthracyclines include: Actinomycin-D, Bleomycin, Mitomycin-C, and Mitoxantrone (also acts as a topoisomerase II inhibitor).

Topoisomerase Inhibitors

These drugs interfere with enzymes called topoisomerases, which help separate the strands of DNA so they can be copied during the S phase. (Enzymes are proteins that cause chemical reactions in living cells.) Topoisomerase inhibitors are used to treat certain leukemias, as well as lung, ovarian, gastrointestinal, and other cancers. Topoisomerase inhibitors are grouped according to which type of enzyme they affect.

Topoisomerase I inhibitors include Topotecan and Irinotecan (CPT-11). Topoisomerase II inhibitors include Etoposide (VP-16), Teniposide and Mitoxantrone (which also acts as an anti-tumor antibiotic). Topoisomerase II inhibitors can increase the risk of a second cancer—acute myelogenous leukemia (AML)—as early as 2 to 3 years after the drug is given.

Mitotic Inhibitors

Mitotic inhibitors are often plant alkaloids and other compounds derived from natural products. They work by stopping mitosis in the M phase of the cell cycle but can damage cells in all phases by keeping enzymes from making proteins needed for cell reproduction. Examples of mitotic inhibitors include: Taxanes: paclitaxel (Taxol®) and docetaxel (Taxotere®); Epothilones: ixabepilone (Ixempra®); Vinca alkaloids: vinblastine (Velban®), vincristine (Oncovin®), and vinorelbine (Navelbine®); and Estramustine (Emcyt®). They are used to treat many different types of cancer including breast, lung, myelomas, lymphomas, and leukemias. These drugs may cause nerve damage, which can limit the amount that can be given.

Corticosteroids

Corticosteroids, often simply called steroids, are natural hormones and hormone-like drugs that are useful in the treatment of many types of cancer, as well as other illnesses. When these drugs are used as part of cancer treatment, they are considered chemotherapy drugs. Examples of corticosteroids include Prednisone, Methylprednisolone (Solumedrol®); and Dexamethasone (Decadron®). Steroids are also commonly used to help prevent nausea and vomiting caused by chemotherapy. They are used before chemotherapy to help prevent severe allergic reactions, too.

Other Chemotherapy Drugs

Some chemotherapy drugs act in slightly different ways and do not fit well into any of the other categories. Examples include drugs like L-asparaginase, which is an enzyme, and the proteosome inhibitor bortezomib (Velcade®).

A localized cancer is usually found in the tissue or organ where it began and has not spread to nearby lymph nodes or to other parts of the body, or the spread is limited in scope.

Non-limiting examples of localized cancers include single-lesion skin cancers, solitary pulmonary nodules (single lung tumor), Adrenal Cancer, Anal Cancer, Bile Duct Cancer, Bladder Cancer, Bone Cancer, Brain/CNS Tumors In Adults, Brain/CNS Tumors In Children, Breast Cancer, Breast Cancer In Men, Cancer in Adolescents, Cancer in Children, Cancer in Young Adults, Cancer of Unknown Primary, Castleman Disease, Cervical Cancer, Colon/Rectum Cancer, Endometrial Cancer, Esophagus Cancer, Ewing Family Of Tumors, Eye Cancer, Gallbladder Cancer, Gastrointestinal Carcinoid Tumors, Gastrointestinal Stromal Tumor (GIST), Gestational Trophoblastic Disease, Hodgkin Disease, Kaposi Sarcoma, Kidney Cancer, Laryngeal and Hypopharyngeal Cancer, Leukemia, Liver Cancer, Lung Cancer, Lung Cancer—Non-Small Cell, Lung Cancer—Small Cell, Lung Carcinoid Tumor, Lymphoma, Lymphoma of the Skin, Malignant Mesothelioma, Multiple Myeloma, Myelodysplastic Syndrome, Nasal Cavity and Paranasal Sinus Cancer, Nasopharyngeal Cancer, Neuroblastoma, Non-Hodgkin Lymphoma, Non-Hodgkin Lymphoma In Children, Oral Cavity and Oropharyngeal Cancer, Osteosarcoma, Ovarian Cancer, Pancreatic Cancer, Penile Cancer, Pituitary Tumors, Prostate Cancer, Retinoblastoma, Rhabdomyosarcoma, Salivary Gland Cancer, Sarcoma—Adult Soft Tissue Cancer, Skin Cancer, Skin Cancer—Basal and Squamous Cell, Skin Cancer—Melanoma, Skin Cancer—Merkel Cell, Small Intestine Cancer, Stomach Cancer, Testicular Cancer, Thymus Cancer, Thyroid Cancer, Uterine Sarcoma, Vaginal Cancer, Vulvar Cancer, Waldenstrom Macroglobulinemia, and Wilms Tumor

In particularly preferred embodiments, localized cancers suitable for treatment by tumescent delivery of a chemotherapy drug include Pancreatic cancer, Ovarian cancer, Lung cancer, Breast cancer, Liver cancer, Melanoma, Kidney cancer, Colon cancer, as well as discrete metastatic lesions.

In tumescent delivery of chemotherapy drug, the subcutaneous concentration of the chemotherapy drug achieved is simultaneously: (i) below the threshold for local tissue toxicity while sufficiently concentrated to result in a significant positive local therapeutic effect, and (ii) greater than the maximum subcutaneous interstitial fluid concentration that can be achieved by conventional intravenous delivery or oral delivery of the chemotherapy drug. For example the subcutaneous concentration of the chemotherapy drug achieved is equal to or about 5%, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, 200%, 250%, 300%, 350%, 400%, 450% or 500% greater than the maximum subcutaneous interstitial fluid concentration that can be achieved by conventional intravenous delivery or oral delivery of the chemotherapy drug.

Biologic Drug Delivery

Biologic drugs generally consist of large organic molecules derived from biological sources. Traditional anticancer chemotherapy drugs are akin to weapons of mass destruction that can damage any living cell. An anticancer chemotherapy drug is only therapeutic if it is more toxic to cancer cells than to healthy cells. In contrast, biologic drugs are focused weapons that targets specific pathologic cells or pathologic cellular products. Examples of Biologic drugs include cytokines, chemokines, growth factors, viral antigens, enzymes, hormones, neurotrophins, antibodies, proteins that target specific genes, antibody to a specific antigen.

An important subset of biologic anticancer drugs consists of antibodies that target specific CD antigens or CD markers on malignant cells. Cluster of Differentiation or Classification Determinant (CD) is a protocol for identifying cell surface molecules and for immunopheno-typing of cells. CD molecules can act as receptors or ligands for altering the behavior of the cell or other functions cell adhesion.

Lymph Node Targeted Drug Delivery

Lymph node targeted drug delivery of anticancer medications using tumescent infiltration provides a unique mode of drug delivery. Lymphatic vessels specifically absorb large molecules from interstitial tissue spaces and return these large molecules to the systemic circulation via lymph nodes. Tumescent infiltration drug delivery can target metastatic cancer cells within lymph nodes. For example, a large volume of dilute solution of proteinaceous anti-melanoma drugs, if infiltrated into the subcutaneous tissue around the site of a primary melanoma tumor, will be absorbed into the lymph vessels that drain the primary tumor site and deliver the drugs directly to the lymph nodes which might have trapped metastatic melanoma cells, thus preventing further, more widespread metastases.

Snake Antivenin Delivery

Snake antivenin delivery is another unique application of tumescent infiltration. Snake venom contains multiple large proteins, which have both local and systemic effects. Antivenin contains antibodies to the venomous proteins. Snakebite is painful. Tumescent infiltration of a snake antivenin can, 1) immediately relieve the pain (lidocaine effect), dilute the venom decreasing tissue toxicity, neutralize much of the venom at the site of the bite before it is systemically absorbed. Venom is absorbed via lymphatic vessels. Large molecular antivenin antibodies are also specifically absorbed via lymphatic vessels. Following tumescent infiltration around the site of a snakebite, the antivenin is absorbed into the same lymphatic vessels as the venom. In this fashion, tumescent antivenin can neutralize the venom within lymphatic vessels before the venom reaches the systemic circulation.

Methods of Preventing or Treating Sepsis

Sepsis is a body's overwhelming and life-threatening response to an infection, which can lead to tissue damage, organ failure, and even death. Patients are given a diagnosis of sepsis when they develop clinical signs of infections or systemic inflammation. Sepsis is not diagnosed based on the location of the infection or by the name of the causative microbe. Physicians draw from a list of signs and symptoms in order to make a diagnosis of sepsis, including abnormalities of body temperature, heart rate, respiratory rate, and white blood cell count. For example, sepsis may be diagnosed in a 72-year-old man with pneumonia, fever, and a high white blood cell count, and in a 3-month-old with appendicitis, low body temperature, and a low white blood cell count.

Sepsis is defined as severe when these findings occur in association with signs of organ dysfunction, such as hypoxemia, oliguria, lactic acidosis, elevated liver enzymes, and altered cerebral function. Nearly all victims of severe sepsis require treatment in an intensive care unit for several days or weeks. While most cases of sepsis are associated with disease or injury, many events follow routine, even elective surgery.

Some embodiments disclosed herein relate to prevention or treatment of sepsis in a subject. Tumescent drug delivery can achieve a localized reservoir of a drug, which is present at a relatively high concentration in local interstitial tissues. While the high concentration of drug delivered by the tumescent technique is confined to localized tissues targeted, a lower systemic level of the drug can also be attained, originating from the localized reservoir established by tumescent delivery. For example, antibiotic or anti-inflammatory agents delivered tumescently can provide ongoing systemic levels of the antibiotic or anti-inflammatory agent, which can effectively prevent or treat sepsis in a subject.

It is understood that the examples and embodiments described herein are for illustrative purposes only and that various modifications or changes in light thereof will be suggested to persons skilled in the art and are to be included within the spirit and purview of this application and scope of any appended claims. All figures, tables, and appendices, as well as publications, patents, and patent applications, cited herein are hereby incorporated by reference in their entirety for all purposes.

While the present description sets forth specific details of various embodiments, it will be appreciated that the description is illustrative only and should not be construed in any way as limiting. Furthermore, various applications of such embodiments and modifications thereto, which may occur to those who are skilled in the art, are also encompassed by the general concepts described herein. Each and every feature described herein, and each and every combination of two or more of such features, is included within the scope of the present invention provided that the features included in such a combination are not mutually inconsistent.

In the various embodiments described herein, components can be added, removed, and/or rearranged. Further, the disclosure herein of any particular feature, aspect, method, property, characteristic, quality, attribute, element, or the like in connection with various embodiments can be used in all other embodiments set forth herein. Additionally, it will be recognized that any methods described herein may be practiced using any device suitable for performing the recited steps.

Although these inventions have been disclosed in the context of certain preferred embodiments and examples, it will be understood by those skilled in the art that the present inventions extend beyond the specifically disclosed embodiments to other alternative embodiments and/or uses of the inventions and obvious modifications and equivalents thereof. In addition, while several variations of the inventions have been shown and described in detail, other modifications, which are within the scope of these inventions, will be readily apparent to those of skill in the art based upon this disclosure. It is also contemplated that various combination or sub-combinations of the specific features and aspects of the embodiments may be made and still fall within the scope of the inventions. It should be understood that various features and aspects of the disclosed embodiments can be combined with or substituted for one another in order to form varying modes of the disclosed inventions. Further, the actions of the disclosed processes and methods may be modified in any manner, including by reordering actions and/or inserting additional actions and/or deleting actions. Thus, it is intended that the scope of at least some of the present inventions herein disclosed should not be limited by the particular disclosed embodiments described above. The limitations in the claims are to be interpreted broadly based on the language employed in the claims and not limited to the examples described in the present specification or during the prosecution of the application, which examples are to be construed as non-exclusive.

What is claimed is:

1. A tumescent solution comprising:
(i) a vasoconstrictor;
(ii) a B vitamin, and
(iii) a pharmaceutically acceptable carrier.

2. The tumescent solution according claim 1, wherein the B vitamin is selected from the group consisting of B1 (Thiamine), B2 (Riboflavin), B3 (Niacin), B5 (Pantothenic acid), B6 (Pyridoxine), B7 (Biotin), B9 (Folic acid) and B12 (Cobalamin).

3. An intravenous (IV) fluid bag or container containing the tumescent solution according to claim 1.

4. The tumescent solution according to claim 1, wherein the B vitamin imparts a visible color to the tumescent solution is selected from the group consisting of Vitamin B12 and Vitamin B2.

5. The tumescent solution according to claim 1, further comprising a local anesthetic.

6. The tumescent composition of claim 5, wherein said local anesthetic is lidocaine.

7. The tumescent composition according to claim 1, wherein the B vitamin is vitamin B12.

8. The tumescent composition according to claim 7, wherein the concentration of vitamin B12 is in the range of 1-20 mg/L.

9. The tumescent composition of claim 6, wherein the concentration of lidocaine is approximately 100 mg/L-2,500 mg/L of solution.

10. The tumescent composition of claim 1, wherein said vasoconstrictor is epinephrine.

11. The tumescent composition of claim 10 wherein the concentration of epinephrine is approximately 0.1 mg/L-2.5 mg/L.

12. The tumescent solution according to claim 1, further comprising a drug or therapeutic agent dissolved in the tumescent solution, wherein a tumescent concentration of the drug or therapeutic agent is simultaneously:
1) below a threshold for local, subcutaneous tissue toxicity,
2) above a threshold for positive local therapeutic effect, and
3) above a concentration safely achievable by intravenous (IV), intramuscular (IM) or oral (PO) delivery.

13. The tumescent composition of claim 12, wherein the drug or therapeutic agent is selected from the group consisting of:
(i) a chemotherapy agent,
(ii) an angiogenesis inhibitor,
(iii) an antiviral agent,
(iv) an antifungal agent,
(v) a biologic drug;
(vi) an antiprotozoal drug;
(vii) a selective serotonin reuptake inhibitor (SSRI);
(viii) an anti-seizure medication;
(ix) an antibiotic,
(x) an anti-inflammatory drug; and
(xi) an antivenom or contravenom.

14. A method of subcutaneous delivery of a drug or therapeutic agent to a subject comprising subcutaneously administering to said subject the tumescent solution according to claim 12.

15. The method of claim 14, wherein infiltration of the tumescent solution achieves both prolonged local drug or therapeutic agent concentration within a tumescent subcutaneous tissue as well as a prolonged slow constant systemic absorption of drugs from the tumescent tissue into a systemic circulation.

16. The method of claim 15, wherein a pharmacokinetic profile of the systemic absorption resembles a slow, constant, intravenous (IV) infusion.

17. The method of claim 14, wherein a subcutaneous concentration of the drug or therapeutic agent achieved is from about 1-100 times the maximum subcutaneous interstitial fluid concentration that can be achieved by conventional IV, IM or oral delivery of the drug or therapeutic agent.

18. The method of claim 14, wherein said drug or therapeutic agent reduces neuropathic pain or the risk of developing neuropathic pain.

19. The method of claim 18, wherein said neuropathic pain is selected from the group consisting of postherpetic neuralgia, trigeminal neuralgia, phantom limb pain, diabetic neuropathy, carpal tunnel syndrome, sciatica, degenerative disk disease, spinal cord injury, post-surgical pain and cancer.

20. A method of subcutaneous delivery of a drug or a therapeutic agent to a subject to treat a localized condition, the method comprising: (a) obtaining the tumescent solution according to claim 11, and (b) delivering a mass of the tumescent solution subcutaneously to a localized tissue in the subject, wherein the drug or the therapeutic agent treats the localized condition in the localized tissue, wherein a concentration of the drug or the therapeutic agent subcutaneously delivered exceeds a concentration of the drug or the therapeutic agent that can be safely achieved by intravenous delivery, and wherein an amount of the drug or the therapeutic agent located at an outer boundary of the mass of fluid and available for absorption is less than an amount of the drug or the therapeutic agent located within a central portion of the mass of fluid and virtually isolated from the systemic circulation by virtue of capillary vasoconstriction.

21. The method according to claim 20, wherein the localized condition is selected from the group consisting of acute Herpes zoster pain, acute musculoskeletal pain, acute traumatic bone fractures, acute burns, acute blast injury, acute non-compressible hemorrhagic combat injury, postherpetic neuralgia, trigeminal neuralgia, phantom limb pain, diabetic neuropathy, carpal tunnel syndrome, sciatica, degenerative disk disease, spinal cord injury, post-surgical pain, cancer, a tumor, a surgical site infection, chronic post-operative pain, chronic alcohol consumption neuropathy, diabetic neuropathy, risk of deep vein thrombosis, risk of post-operative thromboembolism and a burn wound.

22. A method of subcutaneous delivery of a drug or a therapeutic agent to a subject to treat a generalized condition, the method comprising:
(a) obtaining the tumescent solution according to claim 1, and
(b) delivering a mass of the tumescent solution subcutaneously to a localized tissue in the subject,
wherein the drug or the therapeutic agent treats a generalized condition or a central nervous system process which affects tissue far beyond in the localized site of the injection.

23. The method of claim 22, wherein the method treats a condition selected from the group consisting of a cerebrovascular accident, multiple sclerosis, and a spinal cord injury.

24. A method of reducing persistent postsurgical pain (PPP) in a subject comprising subcutaneously injecting into a surgical site of the subject a composition according to claim 1.
